(19) United States
(12) Patent Application Publication
(10) Pub. No.: US 2022/0120746 A1
(43) Pub. Date: Apr. 21, 2022

(54) Methods of Determining Susceptibility to COVID-19 Infection

(71) Applicant: Sabine Hazan, Ventura, CA (US)

(72) Inventor: Sabine Hazan, Ventura, CA (US)

(21) Appl. No.: 17/501,250

(22) Filed: Oct. 14, 2021

Related U.S. Application Data
(63) Continuation-in-part of application No. 17/406,774, filed on Aug. 19, 2021.
Application No. 17/406,774 is a continuation-in-part of application No. 17/200,585, filed on Mar. 12, 2021.
Application No. 17/200,585 is a continuation-in-part of application No. 17/026,051, filed on Sep. 18, 2020, now Pat. No. 11,253,534.
(60) Provisional application No. 63/002,486, filed on Mar. 31, 2020.
(60) Provisional application No. 62/991,699, filed on Mar. 19, 2020.
(60) Provisional application No. 62/991,146, filed on Mar. 18, 2020.
(60) Provisional application No. 62/991,190, filed on Mar. 18, 2020.

(51) Int. Cl. G01N 33/569; G01N 33/50; A61P 1/14; A61K 35/745

(52) U.S. Cl. CPC G01N 33/56911; G01N 33/5008; C12R 2001/145; A61K 35/745; G01N 2800/06; A61P 1/14

(57) ABSTRACT

A method of determining susceptibility to COVID-19 infection comprising the steps of providing a stool sample, and determining an amount of Bifidobacterium, Clostridium, Faecalibacterium, Faecalibacterium prausnitzii, Ruminococcus, and Subdoligranulum in the stool sample. A method of determining susceptibility to COVID-19 infection comprising the steps of providing a stool sample, and determining an amount of Bifidobacterium and Faecalibacterium, in the stool sample. A method of reducing the severity of COVID-19 infection comprising the steps of providing the individual, and administering at least one of the following: Bifidobacterium and or Faecalibacterium. A method of reducing the risk of infection of COVID-19 comprising the steps of providing the individual, and administering Bifidobacterium and or Faecalibacterium. A method of increasing an amount of Bifidobacterium and Faecalibacterium in an individual, the method comprising the steps of providing the individual; and administering one or more of the following: Vitamin C, doxycycline, Zinc, and Ivermectin.

### CROSS-REFERENCE TO RELATED APPLICATIONS

This application is a continuation-in-part of U.S. patent application Ser. No. 17/406,774, titled “Methods of Preventing and Treating COVID-19 Infection with Probiotics,” filed Aug. 19, 2021, which is a continuation-in-part of U.S. Non-Provisional patent application Ser. No. 17/200,585, titled “Methods of Preventing and Treating COVID-19 Infection with Probiotics,” filed Mar. 12, 2021, which is a continuation-in-part of United States Non-Provisional patent application Ser. No. 17/026,051, titled “Method for Preventing and Treating COVID-19 Infection,” filed Sep. 18, 2020. This application claims priority to U.S. Provisional Patent Application No. 63/002,486, titled “Method of Analyzing the Microbiome of Individual Stool Samples,” filed Mar. 31, 2020, U.S. Provisional Patent Application Ser. No. 62/991,699, titled “Autologous Gastrointestinal Microbiota Preservation,” filed Mar. 19, 2020, U.S. Provisional Application Ser. No. 62/991,146, titled “Autologous and Familial Fecal Microbiota Transplant,” filed Mar. 18, 2020, and U.S. Provisional Patent Application No. 62/991,190, titled “Method of Analyzing the Microbiome of Individual Stool Samples,” filed Mar. 18, 2020. The contents of all of these applications are incorporated by reference in their entirety.

### BACKGROUND

The human gastrointestinal (GI) microbiome is a complex, interconnected web of microbes, living in a symbiotic relationship with their host. There are greater than ten times more bacteria in the human body than there are human cells, all in a delicate and ever-changing balance to maintain a healthy GI tract. When this balance is disrupted, a condition known as dysbiosis, or disease, can occur. Traditional methods of treating disease and infection include the use of prescription medications, which come with potentially serious side effects and other issues.

COVID-19 is a novel betacoronavirus that originated in bats in the city of Wuhan, China. This disease has rapidly spread to become a worldwide pandemic, as declared by the World Health Organization (WHO). Symptoms of COVID-19, including fever, myalgia, coughing and shortness of breath, may appear from 2 and 14 days after exposure. Approximately 20% of patients progress to severe illness, including pneumonia, respiratory distress, and even death. Cases in the US have increased five-fold over the last week, alone. The disease is spreading rapidly, and a cure is desperately needed.

Thus, there is a significant unmet need for diagnosing susceptibility to COVID-19 infection.

### SUMMARY

The present invention addresses this need. In a first embodiment, the present invention is directed to my method of determining susceptibility to COVID-19 infection by determining an amount of Bifidobacterium, Clostridium, Faecalibacterium, Faecalibacterium prausnitzii, Ruminococcus, and Subdoligranulum in the stool sample. The amounts determined to indicate a susceptibility to COVID-19 infection are a decreased abundance of Bifidobacterium, Clostridium, Faecalibacterium, Faecalibacterium prausnitzii, Ruminococcus, and Subdolingranulum and an increased abundance of Bacteroides, as compared to an individual who is less susceptible to COVID-19 infection.

More specifically, COVID-19 susceptibility can be determined by determining an amount of Bifidobacterium and Faecalibacterium in the stool sample. Susceptibility is indicated by a decreased abundance of Bifidobacterium and Faecalibacterium as compared to an individual who is less susceptible to COVID-19 infection.

Shotgun next-generation sequencing is performed on the stool sample to determine the bacteria levels.

The severity of COVID-19 infection in an individual can be reduced by administering at least one of the following to the individual: Bifidobacterium and or Faecalibacterium, and they can be administered to the individual orally, topically, or anally.

The severity of COVID-19 infection in an individual can be reduced by administering Bifidobacterium, Clostridium, Faecalibacterium, Faecalibacterium prausnitzii, and Ruminococcus.

The risk of COVID-19 infection in an individual can be reduced by administering at least one of the following to the individual: Bifidobacterium and or Faecalibacterium, and they can be administered to the individual orally, topically, or anally.

The risk of COVID-19 infection in an individual can be reduced by administering Bifidobacterium, Clostridium, Faecalibacterium, Faecalibacterium prausnitzii, and Ruminococcus.

The amount of Bifidobacterium and Faecalibacterium in an individual can be increased by administering one or more of the following: Vitamin C, Zinc, Ivermectin and doxycycline. The vitamin C, zinc, ivermectin and doxycycline can be administered to the individual orally, topically, intravenously, submuscularly or anally.

1,000 mg to 14,000 mg of vitamin C can be administered to the individual at least once to increase the amount of Bifidobacterium and Faecalibacterium in the individual.

25 mg to 75 mg of zinc, 200 mg of doxycycline, and 4 mg to 200 mg of ivermectin can be administered to the individual at least once to increase the amount of Bifidobacterium and Faecalibacterium in the individual.

4 mg to 200 mg of ivermectin is administered to the individual at least once to increase the amount of Bifidobacterium and Faecalibacterium in the individual.

### DRAWINGS

These and other features, aspects and advantages of the present invention will be better understood with reference to the following description, appended claims, and accompanying drawings where:

FIG. 1 is a flow chart of a method of preventing COVID-9 infection in an individual by administering probiotics;

FIG. 2 is a flow chart of a method of treating an individual infected with COVID-19 by administering probiotics;

FIG. 3 is a flow chart of a method of treating an individual infected with COVID-19 with fecal microbiota transplant, having features of the present invention;

FIG. 4 is a top plan view of a stool collection kit having features of the present invention;

FIG. 5 is top plan view of the stool collection kit of FIG. 4, wherein the contents have been removed from the box; and

FIG. 6A is a graph showing the Alpha diversity of SARS-CoV-2 positive patients and exposed healthy controls using a Shannon index [P=0.026];

FIG. 6B is a graph showing the Alpha diversity index of SARS-CoV-2 positive patients and exposed healthy controls using a Simpson index [P=0.043];

FIG. 7 is a graph showing the relative abundance of Bifidobacteria for each subject, grouped by COVID-19 severity;

FIG. 8 is a diagram showing a proposed mechanism for cytokine storm and immune hyper-response in SARS-CoV-2 positive patients;

FIG. 9 is a graph showing Bifidobacterium levels before and after Vitamin C administration;

FIG. 10 is a graph showing Bifidobacterium levels before and after administration of vitamin C, vitamin D, zinc, and ivermectin; and

FIG. 11 is a graph showing Bifidobacterium levels before and after ivermectin administration.

### DETAILED DESCRIPTION

The following discussion describes in detail one embodiment of the invention and several variations of that embodiment. This discussion should not be construed, however, as limiting the invention to those particular embodiments. Practitioners skilled in the art will recognize numerous other embodiments as well.

### Definitions

As used herein, the following terms and variations thereof have the meanings given below, unless a different meaning is clearly intended by the context in which such term is used.

The terms “a,” “an,” and “the” and similar referents used herein are to be construed to cover both the singular and the plural unless their usage in context indicates otherwise.

As used in this disclosure, the term “comprise” and variations of the term, such as “comprising” and “comprises,” are not intended to exclude other additives, components, integers, ingredients or steps.

The term “probiotic” as used herein means a probiotic substance or preparation; a microorganism (or combination of microorganisms) introduced into the body for its beneficial qualities.

The term “microorganism” as used herein means a microscopic organism, including a bacterium, virus, or fungus.

Referring now to the drawings, wherein like reference numerals designate identical or corresponding features throughout the several views. Further, described herein are certain non-limiting embodiments of my pipeline filter assembly for pool filtering and maintenance.

The following discussion describes in detail multiple embodiments of the invention with several variations of those embodiments. This discussion should not be construed, however, as limiting the invention to those particular embodiments. Practitioners skilled in the art will recognize numerous other embodiments as well.

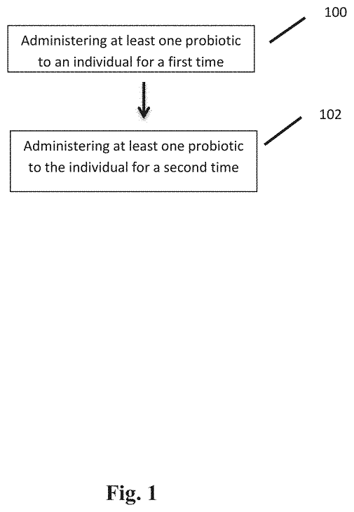

Referring now to FIG. 1, there is shown a first embodiment of the present invention, which is directed to a method of preventing COVID-19 infection in an individual by administering probiotics. The method of prevention comprises the step of administering 100 at least one probiotic to an individual. Optionally, the probiotic can be administered more than one time 102.

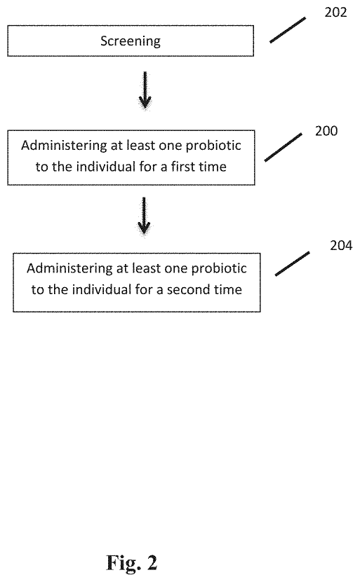

Referring now to FIG. 2, there is shown a second embodiment of the present invention, which is directed to a method of treating COVID-19 infection in an individual by administering probiotics. The method of treatment comprises administering 200 at least one probiotic to an individual infected with COVID-19. Optionally, the individual is first screened 202 for infection with COVID-19 prior to administration of the probiotics. Optionally, the probiotic can be administered more than one time 204.

In both embodiments, the probiotics can comprise any of the bacteria listed in Example 6. More specifically, the probiotics can comprise one or more of the following: Bifidobacterium, Clostridium, Veillonella, Ruminococcus, Sutterella, Faecalibacterium, and Erysiplatoclostridium at the species and genus levels, Bifidobacteriacae, Veilloneacellae, Sutterellacae, Prevotellaceae and Erysipelotrichaceae at the family level, Bifidobacteriales, Veillonellales, Burkholderiales, and Erysipelotrichales at the order level, Actinobacteria, Negativicutes, Betaproteobacteria, and Erysipelotrichia class level and more importantly, Proteobacteria and Actinobacteria at the phylum level. Preferably, the probiotic comprises one or more of the following. Bifidobacterium, Clostridium, Faecalibacterium, Faecalibacterium prausnitzii, Ruminococcus, and Subdoligranulum.

The probiotics can be administered via the following methods: fecal transplant, topical application in the form of a cream, oil, liquid or semi-solid, or orally in the form of a pill/lozenge, liquid tincture or drink, chewable tablet, food stuff such as yogurt, or pressurized spray, or nasogastric tube. The fecal transplant can be administered via colonoscopy, suppository in form of a liquid dosage (e.g., enemas), solid dosage (e.g., suppositories, capsules, and tablets), and semi-solid dosage (e.g., gels, foams, and creams), or topical. The various methods listed above of administering the probiotics are not exclusive of each other. As such, any combination of the above methods of administering the probiotic can be used.

The above listed bacteria can be given to the patient singly (e.g. the probiotic only contains one type of bacteria), or the probiotic can contain one or more of the bacteria listed above plus multiple other microbes like fecal material (meaning as a fecal transplant).

The methods of the present invention can be used to prevent and treat a plurality of diseases, including but not limited to COVID-19/corona virus infection, skin cancer, Clostridioides Difficile Infection. Obesity, Alzheimer's Disease, Crohn's Disease, Myalgic Encephalomyelitis/Chronic Fatigue Syndrome (ME/CFS), Psoriasis, Chronic Urinary Tract Infections, Ulcerative Colitis, Multiple Sclerosis, Chronic Constipation, Lyme Disease, Celiac Disease, Parkinson's Disease, Elevated Cholesterol, Colorectal Cancer, Amyotrophic Lateral Sclerosis (ALS), Fatty Liver, Rheumatoid Arthritis, Anxiety, Obsessive-Compulsive Disorder, Bipolar Disorder, Migraine Headaches, Depression, Diabetes Mellitus, Lupus, Epidermolysis Bullosa, Metastatic Mesothelioma, Eczema, Acne, Irritable Bowel Syndrome, Myasthenia Gravis, Gout, and Autism Spectrum Disorders.

The human gastrointestinal (GI) microbiome is a complex, interconnected web of microbes, living in a symbiotic relationship with their host. There are greater than ten times more bacteria in in the human body than there are human cells in the human body, all in a delicate and ever-changing balance to maintain a healthy GI tract. When this balance is disrupted, a condition known as dysbiosis, or disease, can occur. There is still a debate over whether dysbiosis is a cause of disease or a symptom of it. Naturally, since the microbiome has such a profound impact on human health, including helping humans digest food, make vitamins, and teach human immune cells to recognize pathogens, it plays a vital role in maintaining health. By manipulating the microbiome of patients with disease or infection-induced dysbiosis, the patient's microbiome can be restored to a pre-infection state.

The present invention accomplishes this restoration by administering probiotics via one of the three methods outlined above. One of the methods of administration is fecal microbiota transplant. The transplants can autologous, meaning utilizing the patient's own stool, collected and stored prior to infection, familial, meaning utilizing a family members stool, or third party donor, meaning the stool is collected from a screened, matched donor that is unrelated to the patient.

In general, the method of administering the probiotics via fecal transplants comprises the following steps: screening the individual/patient 300, acquiring a fecal sample from the individual/patient 302, processing the fecal sample from the patient 304, sequencing the fecal sample from the patient 306, analyzing the sequenced fecal sample 308, performing the fecal microbiota transplant 310, and monitoring the patient 312. However, not all of the steps may be required, and the steps of the method of the present invention can vary depending on whether the transplant is autologous, familial, or third party.

Autologous Fecal Transplant

The autologous fecal transplant method comprises three main: screening the patient 300, acquiring a sample from the patient 302, and transplanting the patient's own fecal microbiota into the patient 310.

During the step of screening 300, the patient undergoes the following, signing of the consent form, providing their medical history and demographics, having an EKG performed, having their vital signs taken/read, providing their height and weight, and providing the staff with a list of their prior and concomitant medications. Concomitant medications include any form of antibiotics, probiotics, or opiates.

The doctor or staff overseeing the procedure verifies all inclusion and no exclusion criteria are met.

The patient may continue to take medications currently prescribed; however, the patient must be able to discontinue antibiotics prior to fecal microbiota transplant.

The patient must agree to discontinue use of outside probiotics; however, consumption of active culture yogurt is permissible.

The patient must agree to utilize either a barrier contraception method with spermicide or an IUD (intra-uterine device) for the duration of the study.

The exclusion criteria comprise the following:

The patient refuses to sign the informed consent form.

The patient has a history of total colectomy with ileorectal anastomosis or proctocolectomy.

The patient has a postoperative stoma, ostomy, or ileoanal pouch.

The patient has short bowel syndrome.

The patient is scheduled for a bowel resection.

The patient has had a bowel perforation within six months of screening.

The patient has known symptomatic obstructive strictures.

The patient was exposed to oral or parenteral antibiotics in the four weeks prior to screening, with the exception of topical antibiotics, which are permitted.

The patient has a positive serology for Hepatitis B, Hepatitis C, or HIV.

The patient is currently diagnosed with, or has a history of, uveitis diagnosed by an optometrist or an ophthalmologist.

The patient has a history of malignancy in the last five years, excluding basal cell carcinoma of the skin or carcinoma in situ of the cervix that has been treated with no evidence of recurrence.

The patient has undergone treatment with total parenteral nutrition.

The patient has a history of active tuberculosis requiring treatment in the past three years.

The patient has a history of drug or alcohol abuse within the past three years.

The patient is a female who is pregnant, intends to become pregnant, or is lactating. This is due to unknown fetal or child effects.

The patient has an inability to adequately communicate with the investigator or their respective designee and/or comply with the requirements of the entire study.

The patient participated in any experimental drug protocol within the past twelve weeks.

The patient has clinically significant abnormalities in hematology or biochemistry as confirmed by repeat testing based on investigator's discretion.

Bloodwork is also performed on the patient, which includes the following: a blood mycobacterial culture, which is a test to look for the bacteria that cause tuberculosis and other infections caused by similar bacteria, a complete blood count, a chemistry panel, and a C-reactive protein test. C-reactive protein (CPR) is a blood test marker for inflammation in the body. CRP is produced in the liver and its level is measured by testing the blood. CRP is classified as an acute phase reactant, which means that its levels will rise in response to inflammation.

The patient undergoes a urinalysis, a fecal calprotectin test, and uveitis screening.

Fecal calprotectin is a biochemical measurement of the protein calprotectin in the stool. Elevated fecal calprotectin indicates the migration of neutrophils to the intestinal mucosa, which occurs during intestinal inflammation, including inflammation caused by inflammatory bowel disease.

Uveitis is a form of eye inflammation. It affects the middle layer of tissue in the eye wall (uvea). Uveitis warning signs often come on suddenly and get worse quickly. Warning signs include eye redness, pain and blurred vision. The condition can affect one or both eyes.

Once all the above has been performed by the doctor/provided by the patient, the doctor provides the patient with at least one stool collection kits and instructs the patient on use of the stool collection kits.

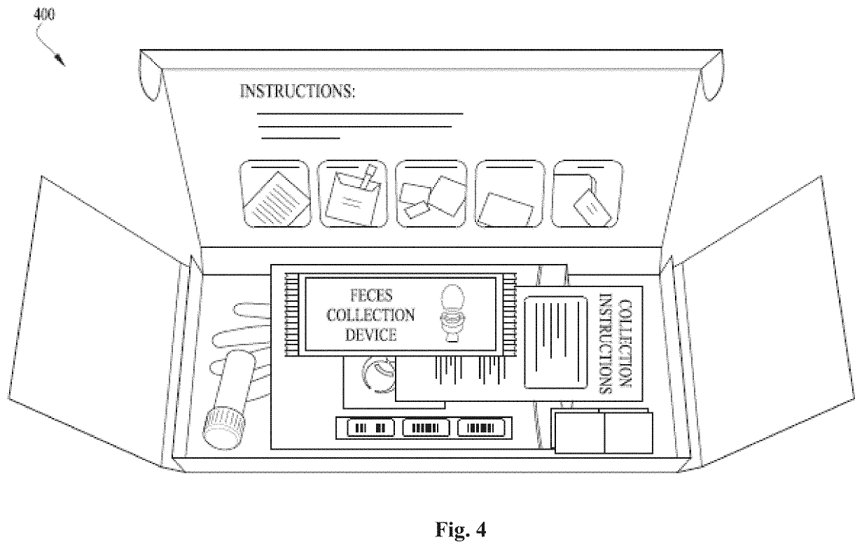

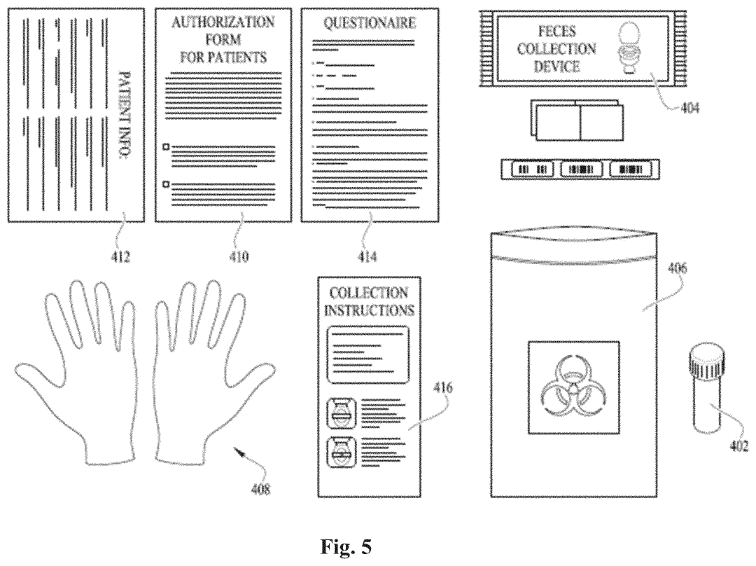

During the step of acquiring a sample 302, regardless of the disease from which the patient is suffering, the following takes place: The step of acquiring a stool sample 302 can either involve the stool sample collection kit 400 or a colonoscopy. The stool sample collection kit 400 is shown in FIGS. 4 and 5 and comprises: at least one stool sample collection vial 402, optionally the vial 402 contains a spoon, at least one toilet accessory or seat cover 404, at least one specimen bag 406, at least one pair of gloves 408, an authorization form 410, a patient information card 412, a questionnaire 414, and stool sample collection instructions 416.

The toilet accessory 404 is in the form of a circular strip of paper that slips over the toilet seat and creates a raised platform on which to provide the voided stool sample.

The stool sample collection instructions 416 are as follows: (1) Correctly position the toilet accessory (i.e. toilet cover) over the toilet seat and put on disposable latex gloves. (2) Unscrew the collection tube cap and use the spoon to scoop one spoonful of the stool sample from the feces. Do not pass the stool sample into the toilet or directly into the collection vial, and do not mix urine or water with the stool sample. (3) Place the stool sample into the collection vial. (4) Tighten the cap and shake to mix the contents thoroughly (and/or invert 10 times) to create a suspension. Some fecal material may be difficult to re-suspend. As long as the stool sample is suspended, the sample is stabilized. Foaming/frothing during shaking is normal. (5) Place the collection vial in the bag labeled “Specimen Bag-Biohazard” and seal the bag. (6) Place the bag back in the collection kit box. (7) Remove toilet cover and let it fall into the toilet bowl. Flush both the toilet cover and excess stool down the toilet. (8) Remove and dispose of gloves. Thoroughly wash hands.

The sample is then processed 304, the microbiome of the sample is sequenced 306 and analyzed 308, and the sample is stored for future use.

In summary, the stool is processed 304 via the following steps:

First, a stool is collected from the patient via an anaerobic method as noted above so that the stool is not exposed to air.

Second, the stool is processed anaerobically in a blender in an anaerobic chamber with normal saline.

Third, the container that the stool is in is kept sealed and stored in a freezer at minus 80 degrees F. until needed for a transplant.

Preferably, the stool sample is stored in a facility much like a sperm bank where individuals can donate their individual stool samples for storage. Most preferably, although not always possible, the individual donates a stool sample (healthy baseline microbiome sample) at birth or at an early stage in life. Optionally, the baseline sample is provided later in life, but pre-hospitalization or pre-epidemic, when the individual is in a healthy state. The purpose is to bank a sample of the individual's healthy microbiome in the event the individual becomes unhealthy and requires a fecal transplant utilizing the healthy sample to reestablish a healthy microbiome. Re-establishing a healthy microbiome will assist the individual in overcoming the current disease, infection, or epidemic.

A more detailed discussion of the processing 304 and sequencing 306 steps are as follows. For these two steps, the following equipment is utilized: centrifuges, pipettes, thermocycler, fluorometers, vortexers, refrigerators/freezers, and a sequencing system (for example, an Illumina NextSeq 550 Sequencing System).

The step of processing the sample 104 includes extracting and purifying patient DNA from the sample. Individual patient DNA is extracted and purified with a DNA extraction kit. Optionally, the QIAmp® PowerFecal® Pro DNA Kit can be used. The DNA extraction kit isolates both microbial and host genomic DNA from stool and gut samples.

In summary, for the DNA extraction step, the stool samples are added to a bead beating tube for rapid and thorough homogenization. Cell lysis occurs by mechanical and chemical methods. Total genomic DNA is captured on a silica membrane in a spin-column format. DNA is then washed and eluted from the membrane and ready for NGS, PCR and other downstream application.

Once the DNA has been extracted, the DNA is then quantitated using a fluorometer. The fluorometer can be a dual-channel fluorometer for nucleic acid quantitation. It provides highly sensitive fluorescent detection when quantifying nucleic acids and proteins.

The following steps are performed when quantitating the sample:

Mix 1-20 microliters of the extracted DNA sample and 200 microliters of dye in a 0.5 ml PCR tube. Mix well by pipetting or vortexing.

The fluorescence is then measured and the nucleic acid concentration is calculated and/or displayed.

Next, the library is prepared. The assay of the present invention is designed to detect all bacteria, viruses, and fungi that reside in the microbiome of the stool samples being evaluated. The assay utilizes an enzymatic reaction to fragment the DNA and to add adapter sequences. Library fabrication includes tagmentation, tagmentation clean-up, and an amplification step followed by another clean-up prior to pooling and sequencing.

The following definitions and abbreviations are used in this section:

BLT: Bead-Linked Transposomes

DNA: Deoxyribonucleic Acid

EPM: Enhanced PCR Mix

EtOH: Ethanol

NGS: Next Generation Sequencing

NTC: No Template Control

PCR: Polymerase Chain Reaction

RSB: Resuspension Buffer

SPB: Sample Purification Beads

TB1: Tagmentation Buffer

TSB: Tagment Stop Buffer

TWB: Tagment Wash Buffer

First, the BLT and TB1 are brought up to room temperature. Then, the BLT and TB1 are vortexed to mix.

Next, the appropriate volume of DNA is added to each well so that the total input amount is 100 nanograms. The optimal input for this assay is 100 nanograms, however, less DNA input can be utilized.

Next, the appropriate volume of nuclease-free water is added to the DNA samples to bring the total volume to 30 microliters.

Then, the BLT is vortexed vigorously for 10 seconds. Next, 11 microliters of BLT and 11 microliters of TB1 are combined for each sample, creating a tagmentation mastermix. Overage is included in this volume.

Next, the tagmentation master mix is vortexed and the volume is equally divided into an 8-tube strip.

Next, 20 microliters of the tagmentation master mix is transferred to each well containing a sample.

Then, the plate is sealed with Microseal ‘B’ and placed on a thermo cycler preprogrammed with the TAG program. The thermo cycler has a heated lid at 100° C. and reaction volume set to 50 microliters.

Next, the TAG program is run as shown in Table 1:

TABLE 1

| Cycle Step | Temperature | Time |
|---|---|---|
| Step 1 | 55° C. | 15 minutes |
| Step 2 | 10° C. | ∞ |

Once the TAG program is complete, the plate is removed from the thermo cycler.

Next, the Microseal ‘B’ seal is removed and 10 microliters of TSB is added to each sample.

Next, the plate is sealed with a Microseal ‘B’ and placed on the thermo cycler preprogrammed with the PTC program. The thermo cycler has a heated lid at 100° C.

Next, the PTC program is shown in Table 2:

TABLE 2

| Cycle Step | Temperature | Time |
|---|---|---|
| Step 1 | 37° C. | 15 minutes |
| Step 2 | 10° C. | ∞ |

When the PTC program is complete, the plate is removed from the thermo cycler and placed on a magnetic stand. The plate is left on the magnetic stand for about 3 minutes (as long as it takes for the solution to clear).

Once the solution is clear, the Microseal ‘B’ is removed from the plate and the supernatant is removed and discarded.

Next, the plate is removed from the magnetic stand and about 100 microliters of TWB is added. The sample should be pipetted slowly until the beads are fully re-suspended.

Next, the plate is placed back on the magnetic stand and approximately 3 more minutes pass while the solution clears again.

Once the solution clears, the supernatant is removed and discarded.

Next, the plate is removed from the magnetic stand and about 100 microliters of TWB is added. The sample should be pipetted slowly until the beads are fully re-suspended.

Next, the plate is again placed on the magnetic stand for an additional 3 minutes while the solution clears.

Next, 22 microliters of EPM and 22 microliters of nuclease-free water are combined with each sample to form a PCR mastermix. Overage is included in this volume. The PCR mastermix is vortexed and centrifuged.

With the plate on the magnetic stand, the supernatant is removed and discarded.

Next, the plate is removed from the magnetic stand and 40 microliters of PCR mastermix are immediately added directly onto the beads in each sample well.

The mastermix is immediately pipetted until the beads are fully re-suspended. Alternatively, the plate is sealed and a plate shaker is used at 1600 rpm for 1 minute.

Next, the plate is sealed with a Microseal ‘B’ and centrifuged at 280×g for 3 seconds.

Next, 10 microliters of index adapters are added to each sample in the plate. The plate is then centrifuged at 280×g for 30 seconds.

Next, the plate is placed on the thermo cycler that is preprogrammed with the BLT PCR program (and with lid preheated at 100° C.).

The BLT PCR Program is run as shown in Table 3:

TABLE 3

| Cycle Step | Number of Cycles | Temperature | Time | |
|---|---|---|---|---|
| Step 1 | 1 | 68° C. | 3 | minutes |
| Step 2 | 1 | 98° C. | 3 | minutes |
| Step 3 | 5 | 98° C. | 45 | seconds |
| | | 62° C. | 30 | seconds |
| | | 68° C. | 2 | minutes |
| Step 4 | 1 | 68° C. | 1 | minute |

| | | | |
|---|---|---|---|
| Step 5 | 1 | 10° C. | ∞ |

When BLT PCR program is complete, the plate is removed from the thermo cycler and centrifuged at 280×g for 1 minute.

Next, the plate is placed on the magnetic stand and it takes about 5 minutes for the solution to clear.

Next, about 45 microliters of supernatant are transferred from each well of the PCR plate to the corresponding well of a new midi plate.

Then, the midi plate is vortexed and the SPB is inverted multiple times to re-suspend.

Next, about 40 microliters of nuclease-free water is added to each sample well containing supernatant.

Next, about 45 microliters of SPB is added to each sample well. Each sample well is then mixed.

The plate is then sealed and incubated for 5 minutes at room temperature.

Next, the plate is placed on the magnetic stand and it takes about 5 minutes for the solution to clear.

Next, the SPB is vortexed thoroughly and 15 microliters of SPB is added to each well of a new midi plate.

Then, 125 microliters of supernatant is transferred from each well of the first plate into the corresponding well of the second midi plate containing 15 microliters SPB.

Each well of the second midi plate is then mixed and the first midi plate can be discarded.

The second midi plate is sealed and incubated for 5 minutes at room temperature.

The second midi plate is placed on the magnetic stand and it takes about 5 minutes for the solution to clear.

Next, without disturbing the beads, the supernatant is removed and discarded.

While the midi plate is still on the magnetic stand, 200 microliters of fresh 80% EtOH are added to the plate, without mixing. The plate is then incubated for 30 seconds.

Next, without disturbing the beads, the supernatant is removed and discarded.

While the second midi plate is still on the magnetic stand, about 200 microliters of fresh 80% EtOH are added, without mixing. The plate is then incubated for 30 seconds.

Next, without disturbing the beads, the supernatant is removed and discarded. Any residual EtOH is also removed and the second midi plate is allowed to air dry on the magnetic stand for about 5 minutes.

The second midi plate is removed from the magnetic stand and about 32 microliters of RSB is added to the beads.

The second midi plate is then re-suspended and incubated for about 2 minutes at room temperature.

The second midi plate is placed back on the magnetic stand it takes about 2 minutes for the solution to clear.

Once the solution clears, about 30 microliters of supernatant are transferred to a new 96-well PCR plate.

Next, the library is pooled and sequenced.

The following definitions and abbreviations are used in this section:

DNA: Deoxyribonucleic Acid

EtOH: Ethanol

HT1: Hybridization Buffer

NGS: Next Generation Sequencing

NTC: No Template Control

RSB: Resuspension Buffer

SAV: Sequencing Analysis Viewer

The following steps are taken to sequence the DNA 106:

1. Prepare the reagent cartridge for use.

2. Denature and dilute sample libraries.

3. Load pooled sample DNA libraries into the prepared reagent cartridge.

4. Set up and start the DNA sequencing using the selected DNA sequencing machine. The sequencing run can take approximately 27-30 hours to complete.

The bioinformatics pipeline utilizes a computational tool that profiles the microbial communities from metagenomic sequencing data with species level resolution. Patient microbiome profiles are analyzed to ascertain not only the profile of microbes in patient samples but also to identify specific strains, and provide accurate estimation of organismal abundance relative to the overall diversity

Once the DNA is sequenced, the microbiome the individual patient is analyzed 308. The step of analyzing 308 the microbiome of the individual can include the following: comparing the microbiome of the individual to the microbiome of the individual's mother, comparing the microbiome of the individual to the microbiome of a sibling of the individual, comparing the microbiome of the individual with a health condition to the microbiome of another individual with same health condition, and comparing the microbiome of the individual with a health condition to the microbiome of the individual before they acquired the health condition (otherwise referred to as baseline versus non-baseline).

If the individual's baseline microbiome is being used in the analysis step 308, then the above recited steps of acquiring a stool sample 302, processing the stool sample 304, and sequencing the microbiome of the individual 306 are performed at least twice—once before the individual acquires a health condition (known as a baseline) and at least once after the individual acquired the health condition. This is necessary so that the baseline microbiome can be compared to the microbiome when the individual is suffering from a health condition.

Optionally, the steps of acquiring a stool sample 302, processing the stool sample 304, and sequencing the microbiome of the individual 306 are performed for a third time, after the individual has overcome the health condition, to confirm that the individual is healthy again.

During the step of transplanting fecal microbiota 310, the following takes place: When the patient suspects they are out of remission, from whatever disease they are suffering from, they return to the doctor's office for the following: vital signs are taken/read, an EKG is performed, their height and weight is recorded, they provide the doctor with an updated list of prior and concomitant medications, and their bloodwork is also repeated. A urinalysis, a fecal calprotectin test, and a uveitis screening are also performed.

Next, the autologous fecal microbiota transplant is scheduled. The patient is also provided with colonoscopy preparation instructions and a bowel cleanse prescription.

Optionally, prior to performing the transplant 310, the patient receives an antibiotic and/or antiparasitic treatment. The antibiotic can comprise one or more of the following: vancomycin, doxycycline, and xifaxan. The antiparasitic can comprise ivermectin.

The antibiotic/antiparasitic treatment can be for a period of 1 to 10 days or up to 6-weeks, and any length of time in between. The dose of antibiotic/antiparasitic is 250 mg of liquid suspension (formulated in a concentration of 500 mg/6 mL) administered orally every 8 hours.

When the patient returns for the transplant, the patient arrives at the surgical center prepped and fasted. The patient signs the informed consent form, and the fecal microbiota transplant is conducted.

The fecal microbiota transplant 310 comprises the following steps:

First, the stool material from the blender is thawed and the probiotics are added. The resulting mixture is placed in syringes anaerobically.

Second, the patient is brought into a surgical suite and sedated in order to perform a colonoscopy.

Third, the patient is placed in the Trendelenburg position, where the body is laid supine, or flat on their back on a 15-30 degree incline with their feet elevated above their head.

Fourth, the mixture of fecal material and probiotics is injected into the cecum.

Fifth, the patient is given atropine or diphenoxylate and loperamide for one week to slow the colon so that the stool material will remain inside the digestive tract.

And finally, if a colonoscopy cannot be performed then the blended stool material mixed with the probiotics is administered to the patient via a nasogastric tube or placed in capsules that patient swallows and are then dissolved in cecum.

Once the transplant 310 is completed, the patient is then monitored 312. The step of monitoring 312 involves monitoring the patient for a short period of time before being discharged and returning to the doctor for at least two follow-up visits. The first follow-up visit is typically 28 days after the transplant.

During the first follow-up visit, the patient undergoes a physical examination, their height and weight are recorded, their vital signs are taken/read and an EKG is performed. Additionally, the patient provides the staff with an updated list of prior and concomitant medications, a stool sample is collected for microbiome analysis, and fecal calprotectin is tested.

The second follow-up visit is typically 28 days after the first follow-up visit. During the second follow-up visit the patient undergoes a physical examination, their height and weight are recorded, and the doctor reviews both the microbiome results and the fecal calprotectin result with the patient. The patient provides the doctor with an updated list of prior and concomitant medications. If the microbiome results are satisfactory, stool will be collected for future autologous fecal microbiota transplant to be done every 6 months.

Familial Fecal Transplant

In the event the transplant is a familial transplant, the family member is the stool donor, the individual supplying the stool sample administered to the patient during the fecal microbiota transplant. As such, the family member that is the stool donor undergoes the screening steps 300 outlined above to ensure the family member does not suffer from the disease or infection, and the acquiring a sample steps 302 outlined above.

Preferably, the family member also undergoes the processing 304, sequencing 306, and analyzing 308 steps outlined above to ensure the familial member is a good match for the patient.

Once the familial stool sample has been collected, the patient receiving the sample then undergoes the transplant 310 and monitoring steps 312 outlined above.

Third Party Donor Fecal Transplant

In the event the transplant is a third party donor transplant, the third party is stool donor, the individual that is supplying the stool sample administered to the patient during the fecal microbiota transplant. As such, the third party undergoes both the screening 300 and the acquiring 302 steps outlined above.

Preferably, the third party donor also undergoes the processing 304, sequencing 306, and analyzing 308 steps outlined above to ensure the third party donor is a good match for the patient.

Once the third party donor stool sample has been collected, the patient receiving the sample then undergoes the transplant 310 and monitoring 312 steps outlined above.

Optionally, as discussed above, the probiotics can be administered to the individual via in the form of a suppository, or orally in the form of a pill/lozenge, liquid tincture or drink, chewable tablet, food stuff such as yogurt, or pressurized spray.

In all instances, amount of probiotic, and the amount or dosage of the bacteria contained within the probiotic can vary from patient to patient, depending on the microbiome needs. The probiotics can be administered for as many days as the supervising physician deems necessary.

Results from the Various Transplant Studies:

An individual infected with COVID-19 can be treated by administering a probiotic containing at least one of the following (or any combination of the same): Bifidobacterium, Clostridium. Veillonella, Ruminococcus, and Sutterella.

Optionally, the above listed bacteria can be administered to the individual orally in form of a pill or spray, or rectally, in the form of a fecal transplant, suppository, or cream.

Optionally, the probiotic can further include any bacteria that are part of the actinobacteria phylum.

If the transplant is familial, then the stool sample utilized during the transplant comes from a donor that is not the patient themselves, but rather a genetically related family member. With respect to the third party donor, the third party donor is not genetically related to the patient.

In both instances, either the family member or the third party donor donates and stores the sample per the protocol described above. When the patient requires a FMT, the appropriate donor sample is selected and administered to the patient. This is considered a matched donor as their microbiome sequences have been “matched.” To achieve successful GR, a detailed patient analysis is performed, as well as meticulous, precise donor selection. There must be a specific match between patient and donor.

Preferably, the donor/third party is genetically related to the patient.

An individual person's microbiome is an accumulation of bacterial DNA, viral DNA, and fungal DNA. The types, quantity and balance of microbes in a person's microbiome are unique to that individual and can affect their susceptibility or resistance to a variety of health issues. The personal microbiome of an individual is as unique to an individual as a fingerprint.

Finding donors is the most critical, and complex, aspect of treatment, and the most important aspect is to precisely match the donor.

Every human being has a unique microbiome in their gut. This is why finding an appropriate donor is extremely complicated and critical to healing. Donor match is the most important factor because if it is not precise, “the same thing that cures a disease can also cause disease.”

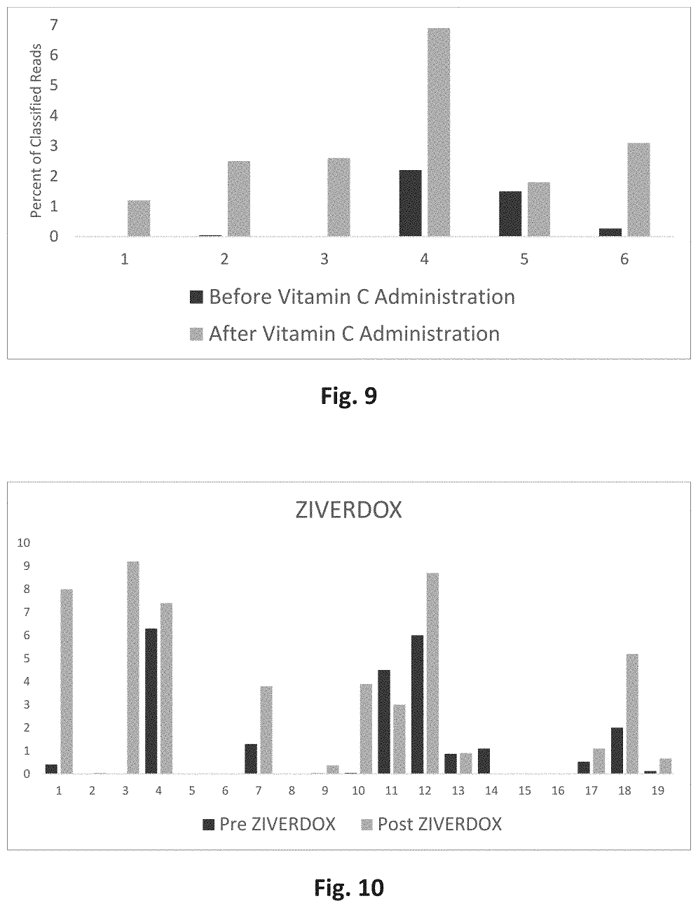

It has been determined that administration of Vitamin C significantly increases Bifidobacterium level, which is shown in FIG. 9. There were 6 patients that were given escalating doses of vitamin C. The doses ranged from 1000 mg up to 14,000 mg, taken by mouth, twice a day, or 1,000 mg to 50,000 mg administered intravenously (IV), and were given from one to ten days.

It has been determined that administration of zinc, ivermectin and doxycycline increase Bifidobacterium levels, which is shown in FIG. 10. There were a total of 19 patients that were given escalating doses of zinc, ivermectin, and doxycycline. The zinc dose ranged from 25 mg to 75 mg a day, the doxycycline dose was 100 mg twice a day, and the ivermectin dose range was from 4 mg to 200 mg a day. All medications were administered for one to ten days.

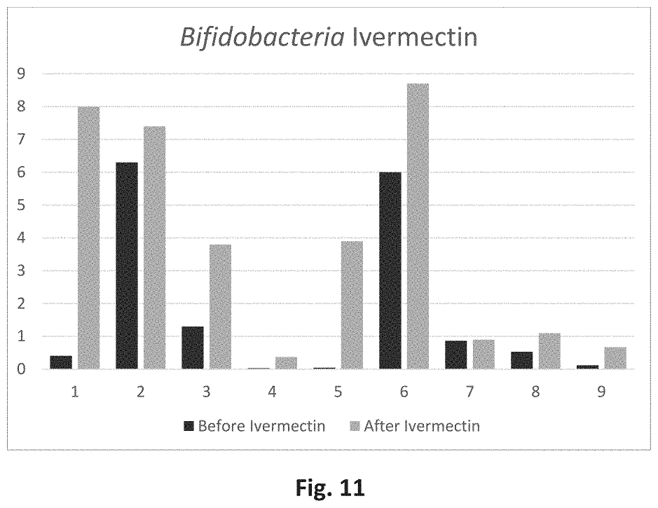

It has been determined that administration of ivermectin alone, increases Bifidobacterium levels, which is shown in FIG. 11. The ivermectin was administered to 9 patients via escalating doses, in a dosage range of 4 mg to 200 mg, orally, twice a day, for one to ten days.

It should be noted that all protocols listed above, including but not limited to the vitamin C, ZIVERDOX (zinc, ivermectin and doxycycline), and ivermectin alone, can be administered orally (including sublingually), rectally, topically (including via the nasal passages as a nasal spray), sub-muscularly, or intravenously.

### EXAMPLES

### Example 1: Vancomycin and Familial Fecal Microbiota Transplant for the Treatment of Subjects with Crohn's Disease

Objectives: Improve the core features of Crohn's disease, including the short and long-term effects of the outcomes; Assess gastrointestinal microbiome relative abundance before and after familial fecal microbiota transplant using whole genome shotgun sequencing; Assess safety and tolerability of familial fecal microbiota transplant in subjects with Crohn's disease.

Procedure: Patients with Crohn's disease are treated with vancomycin followed by familial fecal microbiota transplant (FFMT). The vancomycin is prescribed at the second baseline visit and is given for 10 days. For FFMT, the donor provides a fresh stool sample the morning of the procedure. The sample is brought by the donor to the study site for processing. Processing includes emulsification with sterile normal saline at a 1:1 ratio to create a fecal slurry.

Following the FFMT by colonoscopy the caregiver is taught how to prepare and administer the FFMT enemas at home (during the week 4 visit). Table 4 documents the treatment protocol.

TABLE 4

| | Intervention Name | |
|---|---|---|
| | Vancomycin | Familial Fecal Microbiota Transplant |
| Dose | Suspension of | Emulsification |
| Formulation | 83 mg/mL | |
| | (500 mg/6 mL) | |
| Route of | Oral | Delivered to the colon directly by |
| Administration | | colonoscope in the first transplant, |
| | | and by enema in all subsequent |
| | | transplants |

Statistical Analysis: The statistical evaluation is performed by an outside statistician using SAS® version 9.3 or higher (Statistical Analysis System, SAS Inc., Cary, N.C.) software package. Descriptive summaries are given by treatment group and/or overall. The number of subjects within each treatment group of the analysis set is given in each table. Categorical variables are summarized with counts (n) and percentages (%), together with the number of non-missing values. Fisher's exact test for categorical variables is used. The number of non-missing values is used as the denominator for the calculation of percentages. Incidence of adverse events is based on the number of subjects in the respective analysis set and treatment group. Descriptive statistics for continuous variables are comprised of the number of non-missing observations (n), mean, standard deviation (SD), median, minimum (Min) and maximum (Max), if not otherwise stated. Student's t-test and Mann-Whitney for continuous parametric and nonparametric variables are used respectively. ANOVA is used for comparing means between individual groups. When applicable, these summaries are provided by visit. In case of premature withdrawal from the trial, efficacy and safety assessments performed at the time point of withdrawal, are summarized, separately to the planned visits. A P-value <0.05 is taken as significant for all analyses.

Protocol-Required Safety Laboratory Assessment is documented in Table 5.

TABLE 5

| Laboratory | | | | |
|---|---|---|---|---|
| Assessments | Parameters | | | |
| Hematology | Platelet count | | RBC indices: | White blood cell |
| | Red blood cell (RBC) Count | | MCV | (WBC) count with |
| | Hemoglobin | | MCH | Differential: |
| | Hematocrit | | % Reticulocytes | Neutrophils |
| | | | | Lymphocytes |
| | | | | Monocytes |
| | | | | Eosinophils |
| | | | | Basophils |
| Clinical Chemistry* | Blood urea | Potassium | Asparatate | Total and direct |
| | nitrogen (BUN) | | Aminotransferase (AST)/ | bilirubin |
| | | | Serum Glutamic- | |
| | | | Oxaloacetic | |
| | | | Transaminase (SGOT) | |
| | Creatinine | Sodium | Alanine | Total protein |
| | | | Aminotransferase (ALT)/ | |
| | | | Serum Glutamic-Pyruvic | |
| | | | Transaminase (SGPT) | |
| | Glucose (fasting) | Calcium | Alkaline phosphatase | |

| | |
|---|---|
| Other Screening | Microbiome analysis of stool samples, infectious agent screening of blood and |
| Tests | stool samples of donor and recipient (See Section 4.1, Paragraph Donor Screening) |

Notes:
*if INR measured which may indicate severe liver injury (possible Hy's Law), must be reported as an SAE

### Example 2: Vancomycin and Familial Fecal Microbiota Transplant for the Treatment of Subjects with Alzheimer's Disease

Objectives: Improve the core features of Alzheimer's Disease and the short and long-term effects of outcomes; Improve the quality of life of subjects and their caregivers; Assess gastrointestinal microbiome relative abundance before and after familial fecal microbiota transplant using whole genome shotgun sequencing

Procedure: Participants in the study are required to meet the clinician on fifteen occasions, two appointments for screening and baseline assessment, as well as to receive prescription for vancomycin, one appointment for familial fecal microbiota transplant (FFMT) by colonoscopy and twelve appointments to study sites for the post-trial assessment.

Participants in the study are required to meet the clinician on fifteen occasions, two appointments for screening and baseline assessment, as well as to receive prescription for vancomycin, one appointment for familial fecal microbiota transplant (FFMT) by colonoscopy and twelve appointments to study sites for the post-trial assessment.

Familial Fecal Microbiota Transplantation by Colonoscopy: After the baseline data have been collected and the inclusion and exclusion criteria verified, the patient will be scheduled for familial fecal microbiota transplantation (FFMT) at an outpatient surgical center. This transplant will utilize donor stool from a first degree relative (sibling or child)

The patient will prepare for the procedure the day prior by drinking a prescribed regimen of bowel cleanse solution and water. The patent will present for the procedure having fasted and prepped as instructed by the investigator. The patient will undergo the FFMT procedure under anesthesia and be driven home afterwards. Under no circumstances is the patient to drive themselves home.

Familial Fecal Microbiota Transplant by Enema: After 4 weeks the patient will begin FFMT enemas at home utilizing fresh stool from the same donor as the first FFMT. The patient will lie in the lateral decubitus position and the enema will be inserted into the anus. The fecal material will be slowly expelled into the patient's rectum. The patient will remain in the lateral decubitus position for approximately 30 minutes before getting up. The patient may then use the restroom.

Post Treatment Assessment: After FFMT by colonoscopy the patient will be called for post treatment follow-up visits monthly for the Alzheimer's disease assessments MMSE and Qol AD. The patient will also bring a fresh stool sample for microbiome analysis to these appointments. At the 3-month, 6-month, 9-month, and 12-month follow-up visits the patient will be assessed by following parameters: physical examination, vital signs, Adverse Events, concomitant medications, MMSE and Qol AD. The patient will have blood drawn for laboratory analysis and will bring a fresh stool sample for microbiome analysis. Table 6 documents the treatment protocol.

TABLE 6

| | Intervention Name | |
|---|---|---|
| | Vancomycin | Familial Fecal Microbiota Transplant |
| Dose | Suspension of | Emulsification |
| Formulation | 83 mg/mL | |
| | (500 mg/6 mL) | |
| Route of | Oral | Delivered to the colon directly by |
| Administration | | colonoscope in the first transplant, |
| | | and by enema in all subsequent |
| | | transplants |

Processing of Sample/Dosing: We will treat patients with AD using vancomycin followed by familial fecal microbiota transplant. The vancomycin will be prescribed at the second baseline visit and will be given for 10 days. This is an open-label study, so blinding is not a component. For FFMT the donor provides a fresh stool sample the morning of the procedure. The sample is brought by the donor to the study site for processing. Processing includes emulsification with sterile normal saline at a 1:1 ratio to create a fecal slurry. Following the FFMT by colonoscopy the caregiver is taught how to prepare and administer the FFMT enemas at home (during the week 4 visit).

Statistical Analysis: The statistical evaluation is performed by an outside statistician using SAS® version 9.3 or higher (Statistical Analysis System, SAS Inc., Cary, N.C.) software package. Descriptive summaries are given by treatment group and/or overall. The number of subjects within each treatment group of the analysis set is given in each table. Categorical variables are summarized with counts (n) and percentages (%), together with the number of non-missing values. Fisher's exact test for categorical variables is used. The number of non-missing values is used as the denominator for the calculation of percentages. Incidence of adverse events is based on the number of subjects in the respective analysis set and treatment group. Descriptive statistics for continuous variables are comprised of the number of non-missing observations (n), mean, standard deviation (SD), median, minimum (Min) and maximum (Max), if not otherwise stated. Student's t-test and Mann-Whitney for continuous parametric and nonparametric variables are used respectively. ANOVA is used for comparing means between individual groups. When applicable, these summaries are provided by visit. In case of premature withdrawal from the trial, efficacy and safety assessments performed at the time point of withdrawal, are summarized, separately to the planned visits. A P-value <0.05 is taken as significant for all analyses.

Protocol-Required Safety Laboratory Assessment is documented in Table 7.

TABLE 7

| Laboratory | | | | |
|---|---|---|---|---|
| Assessments | Parameters | | | |
| Hematology | Platelet count | | RBC indices: | White blood cell |
| | Red blood cell (RBC) Count | | MCV | (WBC) count with |
| | Hemoglobin | | MCH | Differential: |
| | Hematocrit | | % Reticulocytes | Neutrophils |
| | | | | Lymphocytes |
| | | | | Monocytes |
| | | | | Eosinophils |
| | | | | Basophils |
| Clinical Chemistry* | Blood urea | Potassium | Asparatate | Total and direct |
| | nitrogen (BUN) | | Aminotransferase (AST)/ | bilirubin |
| | | | Serum Glutamic- | |
| | | | Oxaloacetic | |
| | | | Transaminase (SGOT) | |
| | Creatinine | Sodium | Alanine | Total protein |
| | | | Aminotransferase (ALT)/ | |
| | | | Serum Glutamic-Pyruvic | |
| | | | Transaminase (SGPT) | |
| | Glucose (fasting) | Calcium | Alkaline phosphatase | |

| | |
|---|---|
| Other Screening | Microbiome analysis of stool samples, infectious agent screening of blood and |
| Tests | stool samples of donor and recipient (See Section 4.1, Paragraph Donor Screening) |

Notes:
*if INR measured which may indicate severe liver injury (possible Hy's Law), must be reported as an SAE

### Example 3: Vancomycin and Familial Fecal Microbiota Transplant for the Treatment of Subjects with Autism Spectrum Disorder

Objectives: Improve the core features of Autism Spectrum Disorder (ASD) (social interaction, communication and behavioral problems) as well as the short and long-term effects of the outcomes; Improve other non-core aspects of behavior or function such as self-injurious behavior; Improve the quality of life of subjects and their caregivers; And assess gastrointestinal microbiome relative abundance before and after familial fecal microbiota transplant using whole genome shotgun sequencing.

Procedure: This is an open-label clinical trial to evaluate the benefits of familial fecal microbiota transplant following a 6-week treatment with Vancomycin in minor and adult subjects with ASD for treatment of social deficits and language delays.

Participants in the study are required to meet the clinician on 15 occasions, two appointments for baseline assessment, one appointment for the fecal microbiota transplant procedure by colonoscopy, and twelve appointments to for the post-transplant assessment.

Donor Screening: Once the patient has been deemed eligible and baseline measurements have been collected, a suitable donor will be determined. This donor should be a first-degree relative of the patient (parent, sibling, or child). This donor will present to the clinic for vital signs, physical exam, blood draw for laboratory analysis, and will provide a fresh stool sample for testing. Blood tests will include the following: CBC, complete metabolic profile, CMV IgG, EBV Ab panel, Entamoeba histolytica Ab. Hepatitis A Ab, Hepatitis B core Ab, Hepatitis B surface Ab, Hepatitis C Ab, HHV-6 IgG, HIV antibody, HSV 1 & 2 IgG, HTLV-I/II Ab, IgE, Immunoglobulins panel QT IgM. IgG, IgA, JC virus Ab, Lymphocyte subset panel I. Strongyloides stercoralis, and Syphilis serology. Stool tests will include: CRE, ESBLs, GI panel by PCR, H. pylori, and VRE.

Processing of Sample/Dosing: We will treat minors and adults with ASD using vancomycine followed by familial fecal microbiota transplant. The vancomycin will be prescribed at the second baseline visit and will be given for six weeks. This is an open-label study, so blinding is not a component. For FFMT the donor will provide a fresh stool sample the morning of the procedure. This will be brought by the donor to the study site for processing. Processing includes emulsification with sterile normal saline at a 1:1 ratio to create a fecal slurry. Following the FFMT by colonoscopy the caregiver will be taught how to prepare and administer the FFMT enemas at home (during the week 4 visit).

Vancomycin Treatment: Patient will be given a course of vancomycin of 6-week duration. The dose will 250 mg of liquid suspension (formulated in a concentration of 500 mg/6 mL) every 8 hours.

Familial Fecal Microbiota Transplantation by Colonoscopy: The patient will be scheduled for familial fecal microbiota transplantation (FFMT) at an outpatient surgical center, to be conducted the day after completion of the Vancomycin treatment. The patient will prepare for the procedure the day prior by drinking a prescribed regimen of bowel cleanse solution and water. The patent will present for the procedure having fasted and prepped as instructed by the investigator. The patient will undergo the FFMT procedure under anesthesia and be driven home afterwards. Under no circumstances is the patient to drive themselves home.

Familial Fecal Microbiota Transplant by Enema: After 4 weeks the patient will begin FFMT enemas at home utilizing fresh stool from the same donor as the first FFMT. The patient will lie in the lateral decubitus position and the enema will be inserted into the anus. The fecal material will be slowly expelled into the patient's rectum. The patient will remain in the lateral decubitus position for approximately 30 minutes before getting up. The patient may then use the restroom.

Post Treatment Assessment: After FFMT, the patient will be called for monthly post treatment follow-up visits. The patient will bring a fresh stool sample for microbiome testing to each of these visits. The following tests will be administered at these monthly visits: ATEC, CARS-II, CFQL and SRS-II. During the 3, 6, 9, and 12-month follow-up visits the patient will have blood drawn for complete blood count and metabolic panel. Vital signs will be taken, adverse events discussed, and concomitant medications will be updated. At the week 4 visit the patient's family will be shown how to prepare and administer the FFMT by enema. During weeks in which the patient does not have a visit, a phone call will be made to check on the patient, and, starting during week 4, to remind the caregiver to administer the FFMT enema at home).

Table 8 documents the treatment protocol.

TABLE 8

| | Intervention Name | |
|---|---|---|
| | Vancomycin | Familial Fecal Microbiota Transplant |
| Dose | Suspension of | Emulsification |
| Formulation | 83 mg/mL | |
| | (500 mg/6 mL) | |
| Route of | Oral | Delivered to the colon directly by |
| Administration | | colonoscope in the first transplant, |
| | | and by enema in all subsequent |
| | | transplants |

Statistical Analysis: The statistical evaluation is performed by an outside statistician using SAS® version 9.3 or higher (Statistical Analysis System, SAS Inc., Cary, N.C.) software package. Descriptive summaries are given by treatment group and/or overall. The number of subjects within each treatment group of the analysis set is given in each table. Categorical variables are summarized with counts (n) and percentages (%), together with the number of non-missing values. Fisher's exact test for categorical variables is used. The number of non-missing values is used as the denominator for the calculation of percentages. Incidence of adverse events is based on the number of subjects in the respective analysis set and treatment group. Descriptive statistics for continuous variables are comprised of the number of non-missing observations (n), mean, standard deviation (SD), median, minimum (Min) and maximum (Max), if not otherwise stated. Student's t-test and Mann-Whitney for continuous parametric and nonparametric variables are used respectively. ANOVA is used for comparing means between individual groups. When applicable, these summaries are provided by visit. In case of premature withdrawal from the trial, efficacy and safety assessments performed at the time point of withdrawal, are summarized, separately to the planned visits. A P-value <0.05 is taken as significant for all analyses.

### Example 4: Treatment of Over 300 Patients with Familial Fecal Microbiota Transplant

Procedure: Over 300 patients were treated with familial fecal microbiota transplant via the methods outlined above. If the fecal donor member was compatible with the patient, treatment of the patient was successful.

Results: Out of over 300 patients treated, two patients had Rheumatoid arthritis that was healed, one patient had Alzheimer's that improved, two patients had Crohn's disease that improved, and two patients had psoriasis that improved.

### Example 5: Presence of the SARS-CoV-2 by NGS of Fecal Samples

Objective: In view of the large percentage of SARS-CoV-2 detectible by RT-PCR in stools of infected patients, the objective was to identify the presence of the SARS-CoV-2 by NGS of fecal samples from symptomatic study participants positive for SARS-CoV-2 by nasopharyngeal sample RT-PCR, in addition to asymptomatic individuals (with or without prior nasopharyngeal sample RT-PCR). The objective was also to execute whole genome analysis to characterize SARS-CoV-2 mutational variations to identify potentially significant nucleotide changes.

Procedure: Study participants (n=14) underwent testing for SARS-CoV-2 from fecal samples by whole genome enrichment NGS. Following fecal collection (Zymo Research Shield Fecal Collection Tubes), RNA was extracted (Qiagen Allprep Power Viral Kit), reverse transcribed (New England Biolabs NEBNext 1st and 2nd Strand Synthesis Modules), library prepped (Illumina Nextera Flex for Enrichment), enriched (Ilumina Respiratory Virus Oligo Panel), and sequenced on Illumina's NextSeq 550 System. Sequences were then mapped to the SARS-CoV-2 Wuhan-Hu-1 (MN90847.3) complete genome utilizing One Codex's SARS-CoV-2 bioinformatics analysis pipeline. SARS-CoV-2 positive samples were further analyzed for mutational variants that differed from the reference genome. Of the 14 study participants, 12 also had their nasopharyngeal swabs tested for SARS-CoV-2 by RT-PCR.

Results: The results from patients that had their stool samples tested by whole genome enrichment NGS, and their nasopharyngeal swabs tested by RT-PCR for the presence of SARS-CoV-2 were evaluated. Of the 14 study participants, ten were symptomatic and tested positive for SARS-CoV-2 by RT-PCR, two asymptomatic individuals tested negative, and two other asymptomatic individuals did not undergo RT-PCR testing (Table 34). Patients 5 and 7, which tested positive by RT-PCR from nasopharyngeal swabs, were treated with the protocol from Example 5 above (Hydroxychloroquine, Azithromycin, vitamin C, vitamin D, and zinc for 10 days prior to fecal collection). Similarly, after positive nasopharyngeal swab, patient 13 was treated with vitamin C, vitamin D, and zinc for 10 days (the same protocol as noted above in Example 5) before fecal collection. The concordance of SARS-CoV-2 detection by enrichment NGS from stools among positive non-treated patients tested by RT-PCR nasopharyngeal analysis was 100% (7/7). Patient 8, who did not undergo nasopharyngeal analysis, tested positive for SARS-CoV-2 by NGS. The three patients (5, 7, 13) that received treatment prior to providing fecal samples, all tested negative by NGS. Asymptomatic patients 2 and 9, who tested negative by nasopharyngeal swab, were also negative by NGS, as was asymptomatic patient 14. Table 9 outlines the symptoms and SARS-CoV-2 testing results.

TABLE 9

| | | Nasopharyngeal | | Fecal | Patient |
|---|---|---|---|---|---|
| Sample ID | Symptoms | Swab (RT-PCR) | Treated | (NGS) | Location |
| Patient 1 | febrile, diarrhea, anosmia, O2 sat. <90% | + | no | + | PA |
| Patient 3 | febrile, diarrhea, O2 sat. <90% | + | no | + | CA |
| Patient 4 | febrile, diarrhea, anosmia, O2 sat. <90% | + | no | + | AZ |
| Patient 6 | febrile, cough, anosmia | + | no | + | AZ |
| Patient 8 | none | n/a | no | + | CA |
| Patient 10 | febrile, cough, headache | + | no | + | GA |
| Patient 11 | febrile, cough, headache | + | no | + | GA |
| Patient 12 | febrile, cough | + | no | + | GA |
| Patient 5 | febrile, cough | + | yes | − | CA |
| Patient 7 | febrile, cough | + | yes | − | GA |
| Patient 13 | febrile, cough | + | yes | − | GA |
| Patient 2 | none | − | no | − | CA |
| Patient 9 | none | − | no | − | CA |
| Patient 14 | none | n/a | no | − | CA |

All fecal samples analyzed by enrichment NGS from positive patients by RT-PCR, achieved 100% genome coverage of SARS-CoV-2 except for patient 3 which had 45%, and patient 10 which had 93% coverage. Table 10 outlines the enrichment NGS metrics.

TABLE 10

| | Genome | # Variants | Mapped | |
|---|---|---|---|---|
| Sample ID | Coverage | (over 10x) | Reads | Mean Depth |
| Patient 1 | 100% | 11 | 465645 | 1129.8x |
| Patient 3 | 45% | 11 | 5984 | 31.7x |
| Patient 4 | 100% | 9 | 131582 | 318.6x |
| Patient 6 | 100% | 10 | 793603 | 1924.6x |
| Patient 8 | 100% | 10 | 496852 | 1206.7 |
| Patient 10 | 93% | 9 | 5929 | 15.6x |
| Patient 11 | 100% | 10 | 1270734 | 3075.3x |
| Patient 12 | 100% | 10 | 38256 | 92.7x |

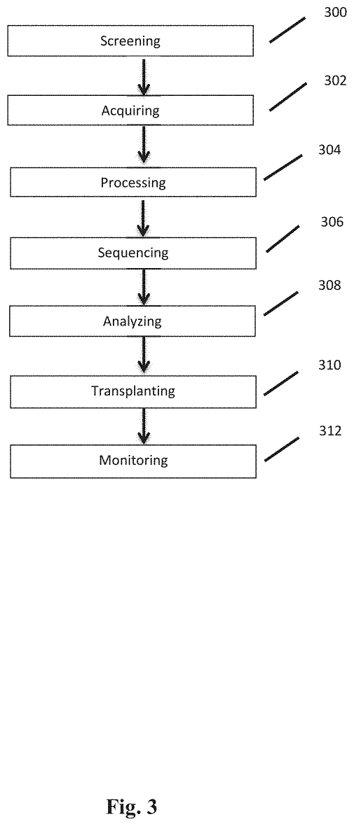

The total number of SARS-CoV-2 mapped reads for patients 1, 3, 4, 6, 8, 10, 11, and 12 were 465645, 5984, 131582, 793603, 496852, 5929, 1270734, and 38256 respectively. The mean read depths of SARS-CoV-2 for patients 1, 3, 4, 6, 8, 10, 11, and 12 were 1129.8x, 31.7x, 318.6x, 1924.6x, 1206.7x, 15.5x, 3075.3x, and 92.7x, and respectively. The read depths at specific coordinates along the SARS-CoV-2 genome for each patient are captured in FIGS. 3A-3H. Whole genome alignment of SARS-CoV-2 in patients 1, 3, 4, 6, 8, 10, 11, and 12 (respectively) as identified by One Codex's SARS-CoV-2 analysis pipeline. The x-axis depicts the genomic coordinates as aligned to the MN908947.3 reference genome, and the y-axis represents the read depth at specific loci.

Following alignment and mapping of SARS-CoV-2, patient genomes were compared to the Wuhan-Hu-1 (MN90847.3) SARS-CoV-2 reference genome via One Codex's bioinformatics pipeline to identify mutational variations. This analysis identified nucleotide variants at positions nt241 (C→T) and nt23403 (A→G) across all positive patients, and variants at positions nt3037 (C→T) and nt25563 (G→T) in seven of the eight patients (Table 3). Interestingly, patients 8, 11, and 12 harbored the same set of variants, as did patients 4 and 6 (who were kindred). Unique variants not identified in any of the other individuals were detected in patients 1, 3, 6, and 10, with patient 3 harboring the most distinct SARS-CoV-2 genome with eight unique variants, followed by patient 1 with seven. Collectively, there were thirty-three different mutations among the patients in which SARS-CoV-2 was detected by whole genome enrichment NGS. Table 11 outlines the SARS-CoV-2 genomic positions, variant changes, and frequencies across the positive patient cohort.

TABLE 11

| Region | | | Patient | Patient | Patient | Patient | Patient | Patient | Patient | Patient |
|---|---|---|---|---|---|---|---|---|---|---|
| (ORF) | Position | Variant | 1 | 3 | 4 | 6 | 8 | 10 | 11 | 12 |
| 5′-UTR | 241 | C → T | 100% | 100% | 100% | 100% | 100% | 100% | 100% | 100% |
| 1a | 833 | T → C | x | x | x | x | 100% | x | 100% | 100% |
| 1a | 1059 | C → T | x | x | 100% | 100% | 99% | 100% | 100% | 100% |
| 1a | 1758 | C → T | x | x | 100% | 100% | x | x | x | x |
| 1a | 1973 | C → T | x | x | x | 87% | x | x | x | x |
| 1a | 3037 | C → T | 100% | x | 100% | 100% | 100% | 100% | 100% | 100% |
| 1a | 3078 | C → T | x | 89% | x | x | x | x | x | x |
| 1a | 4866 | G → T | 75% | x | x | x | x | x | x | x |
| 1a | 6720 | C → T | 93% | x | x | x | x | x | x | x |
| 1a | 8102 | G → T | x | 100% | x | x | x | x | x | x |
| 1a | 9401 | T → C | x | x | x | x | x | 64% | x | x |
| 1a | 9403 | T → A | x | x | x | x | x | 64% | x | x |
| 1a | 10870 | G → T | x | x | 100% | 100% | x | x | x | x |
| 1a | 11123 | G → A | x | x | 100% | 100% | x | x | x | x |
| 1b | 14408 | C → T | 100% | x | 100% | 100% | 100% | x | 100% | 100% |
| 1b | 14877 | C → T | x | 100% | x | x | x | x | x | x |
| 1b | 16616 | C → T | x | x | x | x | 100% | x | 100% | 100% |
| 1b | 16848 | C → T | 100% | x | x | x | x | x | x | x |
| 1b | 18652 | C → A | x | x | x | x | x | 83% | x | x |
| 1b | 19989 | T → G | x | 100% | x | x | x | x | x | x |
| Spike | 21576 | T → G | x | 83% | x | x | x | x | x | x |
| Spike | 23264 | G → A | x | 75% | x | x | x | x | x | x |
| Spike | 23403 | A → G | 100% | 100% | 100% | 100% | 100% | 100% | 100% | 100% |
| Spike | 23603 | C → T | 82% | x | x | x | x | x | x | x |
| 3a | 25563 | G → T | x | 100% | 100% | 100% | 100% | 100% | 100% | 100% |
| 3a | 25976 | C → A | x | x | x | x | 100% | x | 100% | 100% |
| 8 | 27964 | C → T | x | x | x | x | 100% | x | 100% | 100% |
| Nucleoprotein | 28881 | G → A | 100% | x | x | x | x | x | x | x |
| Nucleoprotein | 28882 | G → A | 100% | x | x | x | x | x | x | x |
| Nucleoprotein | 28883 | G → C | 100% | x | x | x | x | x | x | x |
| Nucleoprotein | 28997 | C → T | x | 100% | x | x | x | x | x | x |
| Nucleoprotein | 29019 | A → T | x | 100% | x | x | x | x | x | x |
| Nucleoprotein | 29364 | C → G | x | x | x | x | x | 85% | x | x |

Discussion: Although previous studies have identified SARS-CoV-2 in fecal collections by RT-PCR, this study was able to report whole genome sequencing (WGS) of SARS-CoV-2 from stool samples. SARS-CoV-2 was identified in patients that tested positive by nasopharyngeal swab RT-PCR analysis and unique genomes in 62.5% of the NGS positive patients was observed. The overall homology among the genomes was high (99.97%), with variations identified in the ORF regions 1a, 1b, S, 3a, 8, and N. Of particular interest, was the adenine to guanine change in the S protein at position nt23403 which converts aspartic acid to glycine (D→G).

Conclusion: Next generation sequencing identified the SARS-CoV-2 whole genome sequence in 100% of patients with positive nasopharyngeal RT-PCR and did not detect it in treated patients, or those with negative rt-PCR. These results highlight the importance of metagenomic analysis of the SARS-CoV-2 viral genome.

### Example 6: Study of Microbiome of Patients with COVID Versus Patients Without COVID

36 Covid-19 patients were studied in a cross-sectional study with 14 healthy controls (HC) to identify microbiome diversity by whole genome enrichment NGS. Patient records were included and compared any significant changes on species, genus, family, order, class, and phylum levels of the Covid-19 positive and HC population.

Deep shotgun microbiome sequencing analysis was performed on fecal samples from the 36 Covid-19 positive patients by whole genome enrichment NGS. The observed versus expected rates were primarily reported.

All individuals aged 3 years and older were eligible for inclusion. For each patient, a so-called period of eligibility for study inclusion was defined, which commenced on the latest of the study start date; A patient's period of eligibility ended on the earliest of registration termination; the end of data collection from their practice; or death.

Data Analysis

The differential taxa was conducted between the Gastrointestinal Microbiome of Covid-19 positive and HC relative abundance utilizing One Codex's bioinformatics analysis pipeline. For evaluating any statistical significance of the patient's data at each Classification at phylum, class, order, family, and genus levels, a Fisher's exact test was conducted between the two variables. Statistical analysis was conducted using chi-squared statistics by R version 3.6.1 (2019-07-05). In the statistical analysis, p-value, Confidence interval and Odds ratio were considered for all comparisons.

Results

The study population included 50 patients (36 Covid-19 positives and 14 HC). For the HC population, data from patients after December 2019 were excluded from the study to avoid overlap between any Covid-19 possible positive patients.

The results from the 50 total patients that had their stool samples tested by whole genome enrichment NGS was evaluated. Detailed demographic and summary data, clinical characteristics including total numbers of diagnoses and events were analyzed for the study cohorts, and are included in Table 12.

TABLE 12

| | | | Significant | | | Stool | Sx |
|---|---|---|---|---|---|---|---|
| | | | Past Medical | PCR | Covid-19 | Collection | Severity |
| No | Age | Sex | History | Date | PCR Resuits | Date | at BL |
| 1 | 20 | Female | Hypothyroid, | Jul. 10, 2020 | Positive | Jun. 24, 2020 | Severe |
| | | | Hashimoto, LCH | | | | |
| 2 | 21 | Female | Nothing to | No info | Positive | Apr. 5, 2020 | Moderate |
| | | | report | | | | |
| 3 | 56 | Male | Nothing to | No info | Positive | Apr. 5, 2020 | No info |
| | | | report | | | | |
| 4 | 25 | Female | Asthma, Reflux, | No info | Positive | Mar. 29, 2020 | Mild |
| | | | GI issue | | | | |
| 5 | 25 | Female | No info | No info | Positive | No info | Moderate |
| 6 | 44 | Female | Food | Apr. 8, 2020 | Positive | Apr. 13, 2020 | Severe |
| | | | sensitivities, gut | | | | |
| | | | issues | | | | |
| 7 | | No | No info | No info | Positive | No info | No info |
| | | info | | | | | |
| 8 | 53 | Male | No info | No info | Positive | No info | Severe |
| 9 | 23 | Male | No info | No info | Positive | No info | No info |
| 10 | 19 | Male | Nothing to | Apr. 22, 2020 | Positive | May 6, 2020 | No info |
| | | | report | | | | |
| 11 | 35 | Female | Seizure | No info | Positive | Jun. 23, 2020 | No info |
| 12 | 32 | Male | Nothing to | | Positive | Jun. 26, 2020 | Mild |
| | | | report | | | | |
| 13 | 63 | Female | No info | No info | Positive | Jul. 20, 2020 | No info |
| 14 | No | Male | No info | No info | Positive | Jun. 28, 2020 | No info |
| | info | | | | | | |
| 15 | 61 | Male | Nothing to | No info | Positive | No info | No info |
| | | | report | | | | |
| 16 | 50+ | Male | No info | No info | Positive | No info | No info |
| 17 | 48 | Female | No info | No info | Positive | No info | Severe |
| 18 | No | Female | No info | No info | Positive | Jun. 27, 2020 | No info |
| | info | | | | | | |
| 19 | 70 | Male | No info | No info | Positive | Aug. 5, 2020 | Severe |
| 20 | 56 | Male | Nothing to | No info | Positive | Aug. 10, 2020 | Severe |
| | | | report | | | | |
| 21 | 50+ | Male | No info | No info | Positive | No info | Moderate |
| 22 | 59 | Female | No info | No info | Positive | Sep. 4, 20 | Severe |
| 23 | 35 | Female | No info | No info | Positive | No info | Moderate |
| 24 | 71 | Male | No info | No info | Positive | No info | No info |
| 25 | 58 | Female | No info | No info | Positive | No info | Moderate |
| 26 | 61 | Female | No info | No info | Positive | No info | No info |
| 27 | 55 | Male | Nothing to | | Positive | Oct. 21, 2020 | Severe |
| | | | report | | | | |
| 28 | 66 | Female | No info | No info | Positive | No info | Moderate |
| 29 | No | No | No info | No info | Positive | No info | No info |
| | info | info | | | | | |
| 30 | 66 | Female | No info | No info | Positive | No info | Severe |
| 31 | No | No | No info | No info | Positive | No info | No info |
| | info | info | | | | | |
| 32 | 61 | Female | No info | No info | Positive | No info | Severe |
| 33 | 69 | Male | No info | No info | Positive | No info | No info |
| 34 | No | No | No info | No info | Positive | No info | No info |
| | info | info | | | | | |
| 35 | 40 | Female | No info | No info | Positive | No info | Moderate |
| 36 | No | No | No info | No info | Positive | No info | No info |
| | info | info | | | | | |
| 37 | 6 | Male | Nothing to | Not | Not applicable- | No info | Not |
| | | | report | applicable | Negative | | applicable- |
| | | | | | | | Negative |
| 38 | 6 | Female | Nothing to | Not | Not applicable- | No info | Not |
| | | | report | applicable | Negative | | applicable- |
| | | | | | | | Negative |
| 39 | 3 | Female | Nothing to | Not | Not applicable- | No info | Not |
| | | | report | applicable | Negative | | applicable- |
| | | | | | | | Negative |
| 40 | 3 | Female | Nothing to | Not | Not applicable- | No info | Not |
| | | | report | applicable | Negative | | applicable- |
| | | | | | | | Negative |
| 41 | 11 | Male | Nothing to | Not | Not appiicable- | No info | Not |
| | | | report | applicable | Negative | | applicable- |
| | | | | | | | Negative |
| 42 | 3 | Male | Nothing to | Not | Not applicable- | No info | Not |
| | | | report | applicable | Negative | | applicable- |
| | | | | | | | Negative |
| 43 | 4 | Female | Nothing to | Not | Not applicable- | No info | Not |
| | | | report | applicable | Negative | | applicable- |
| | | | | | | | Negative |
| 44 | 7 | Female | Nothing to | Not | Not appiicable- | No info | Not |
| | | | report | applicable | Negative | | applicable- |
| | | | | | | | Negative |
| 45 | 16 | Female | Nothing to | Not | Not applicable- | No info | Not |
| | | | report | applicable | Negative | | applicable- |
| | | | | | | | Negative |
| 46 | 9 | Male | Nothing to | Not | Not applicable- | No info | Not |
| | | | report | applicable | Negative | | applicable- |
| | | | | | | | Negative |
| 47 | 10 | Female | Nothing to | Not | Not applicable- | No info | Not |
| | | | report | applicable | Negative | | applicable- |
| | | | | | | | Negative |
| 48 | 7 | Female | Nothing to | Not | Not applicable- | No info | Not |
| | | | report | applicable | Negative | | applicable- |
| | | | | | | | Negative |
| 49 | 55 | Female | Nothing to | Not | Not applicable- | No info | Not |
| | | | report | applicable | Negative | | applicable- |
| | | | | | | | Negative |
| 50 | 17 | Female | Nothing to | Not | Not applicable- | No info | Not |
| | | | report | applicable | Negative | | applicable- |
| | | | | | | | Negative |

Tables 13-18 demonstrate the association between relative abundance of the gastrointestinal microbiome at Phylum, Class, Order, Family and Species level of Covid-19 positive (Covid +) vs. healthy control (Covid −) patients. A 95% Confidence interval (CI) and odds ratio (OR) were used in addition to the p-value for better comparison.

TABLE 13

| | | | | | | Fisher exact lest value |
|---|---|---|---|---|---|---|
| | | | | | Statistical | with 95% Confidence |
| Classification | Covid+ Patients | | Covid− Patients | | Significance | Intervals(CI) and odds |
| Phylum | Presence | Absence | Presence | Absence | (P < 0.05) | ratio(OR) |
| Actinobacteria | 31 | 5 | 14 | 0 | Yes* | P < 0.00001(CI; |
| | | | | | | 0.00-0.3, OR = 0) |
| Firmicutes | 36 | 0 | 14 | 0 | No** | p-value 1 |
| Proteobacteria | 33 | 3 | 14 | 0 | Yes | P value 0.003(CI; |
| | | | | | | 0.00-0.48, OR = 0) |
| Bacteroidetes | 36 | 0 | 14 | 0 | No | p-value 1 |

TABLE 14

| | | | | | | Fisher exact test value |
|---|---|---|---|---|---|---|
| | | | | | Statistical | with 95% Confidence |
| Classification | Covid+ Patients | | Covid− Patients | | Significance | Intervals(CI) and odds |
| Class | Presence | Absence | Presence | Absence | (P < 0.05) | ratio(OR) |
| Actinobacteria | 24 | 12 | 14 | 0 | Yes | P < 0.00001(CI; |
| | | | | | | 0.00-0.07, OR = 0) |
| Clostridia | 36 | 0 | 14 | 0 | No | p-value 1 |
| Negativicutes | 30 | 6 | 14 | 0 | Yes | P < 0.00001(CI; |
| | | | | | | 0.00-0.21, OR = 0) |
| Betaproteobacteria | 21 | 15 | 12 | 2 | Yes | P < 0.00001(CI; |
| | | | | | | 0.10-0.46, OR = 0.22) |
| Bacteroidia | 36 | 0 | 14 | 0 | No | p-value 1 |
| Gammaproteobacteria | 21 | 15 | 9 | 5 | No | p-value 0.46(CI; |
| | | | | | | 0.4-1.4, OR = 0.77) |
| Erysipelotrichia | 33 | 3 | 11 | 3 | Yes | p-value 0.009(CI; |
| | | | | | | 01.27-8.77, OR = 3.19) |

TABLE 15

| | | | | | | Fisher exact test value |
|---|---|---|---|---|---|---|
| | | | | | Statistical | with 95% Confidence |
| Classification | Covid+ Patients | | Covid− Patients | | Significance | Intervals(CI) and odds |
| Order | Presence | Absence | Presence | Absence | (P < 0.05) | ratio(OR) |
| Bifidobacteriales | 20 | 16 | 14 | 0 | Yes | P < 0.00001(CI; |
| | | | | | | 0.00-0.05, OR = 0) |
| Clostridiales | 36 | 0 | 14 | 0 | No | p-value 1 |
| Veillonellales | 14 | 22 | 11 | 3 | Yes | P < 0.00001(CI; |
| | | | | | | 0.08-0.30, OR = 0.16) |
| Burkholderiales | 21 | 15 | 12 | 7 | Yes | P < 0.00001(CI; |
| | | | | | | 0.11-0.50, OR = 0.24) |
| Bacteroidales | 36 | 0 | 14 | 0 | No | p-value 1 |
| Erysipelotrichales | 33 | 3 | 14 | 0 | Yes | 0.003(CI; 0.00-0.48, |
| | | | | | | OR = 0) |
| Enterobacterales | 21 | 15 | 6 | 8 | Yes | p-value 0.03(CI; |
| | | | | | | 01.04-3.47, OR = 3.90) |

TABLE 16

| | | | | | | Fisher exact test value |
|---|---|---|---|---|---|---|
| | | | | | Statistical | with 95% Confidence |
| Classification | Covid+ Patients | | Covid− Patients | | Significance | Intervals(CI) and odds |
| Family | Presence | Absence | Presence | Absence | (P < 0.05) | ratio(OR) |
| Bifidobacteriacae | 20 | 16 | 14 | 0 | Yes | P < 0.00001(CI; |
| | | | | | | 0.00-0.05, OR = 0) |
| Clostridiacae | 35 | 1 | 14 | 0 | No | p-value 0.24(CI; |
| | | | | | | 0.0-2.4, OR = 0) |
| Veilloneacellae | 15 | 22 | 11 | 3 | Yes | P < 0.00001(CI; |
| | | | | | | 0.05-0.25, OR = 0.12) |
| Ruminococcacea | 36 | 0 | 14 | 0 | No | p-value 1 |
| Suttereilacae | 19 | 17 | 11 | 3 | Yes | p-value 0.0003(CI; |
| | | | | | | 0.16-20.61, OR = 0.32) |
| Faecalibacterium | 31 | 5 | 14 | 0 | Yes | p-value 0.00007(CI; |
| | | | | | | 0.00-0.27, OR = 0) |
| Prevotellaceae | 17 | 19 | 10 | 4 | Yes | p-value 0.0009(CI; |
| | | | | | | 0.19-0.67, OR = 0.36) |
| Erysipelotrichaceae | 33 | 3 | 14 | 0 | Yes | p-value 0.006(CI; |
| | | | | | | 0.00-0.57, OR = 0) |
| Enterobacteriaceae | 21 | 15 | 8 | 6 | No | p-value 1 |

TABLE 17

| | | | | | Statistical | |
|---|---|---|---|---|---|---|
| Classification | Covid+ Patients | | Covid− Patients | | Significance | |
| Genus | Presence | Absence | Presence | Absence | (P < 0.05) | Fisher exact test value |
| Bifidobacterium | 20 | 16 | 14 | 0 | Yes | P < 0.00001(CI; |
| | | | | | | 0.00-0.05, OR = 0) |
| Clostridium | 33 | 3 | 14 | 0 | Yes | p-value 0.006(CI; |
| | | | | | | 0.00-0.57, OR = 0) |
| Veillonella | 14 | 22 | 11 | 3 | Yes | P < 0.00001(CI; |
| | | | | | | 0.08-0.30 OR = 0.16) |
| Ruminococcus | 30 | 6 | 14 | 0 | Yes | p-value 0.00007(CI; |
| | | | | | | 0.00-0.21, OR = 0) |
| Sutterella | 7 | 29 | 12 | 2 | Yes | P < 0.00001(CI; |
| | | | | | | 0.01-0.08 OR = 0.03) |
| Faecalibacterium | 31 | 5 | 14 | 0 | Yes | p-value 0.00007(CI; |
| | | | | | | 0.00-0.21, OR = 0) |
| Prevotella | 10 | 26 | 3 | 11 | No | p-value 0.40(CI; |
| | | | | | | 0.68-2.83, OR = 1.38) |
| Erysiplatoclostridium | 26 | 10 | 12 | 2 | Yes | p-value 0.03(CI; |
| | | | | | | 0.20-0.96, OR = 0.45) |
| Escherichia | 21 | 15 | 7 | 7 | No | p-value 0.32(CI; |
| | | | | | | 0.76-2.5, OR = 1.37) |
| Klebsiella | 3 | 33 | 1 | 13 | No | p-value 1 |

TABLE 18

| | | | | | Statistical | |
|---|---|---|---|---|---|---|
| Classification | Covid+ Patients | | Covid− Patients | | Significance | |
| Species | Presence | Absence | Presence | Absence | (P < 0.05) | Fisher exact test value |
| Bifidobacterium | 20 | 16 | 14 | 0 | Yes | P < 0.00001(CI; |
| | | | | | | 0.00-0.05, OR = 0) |
| Clostridium | 33 | 3 | 14 | 0 | Yes | p-value 0.006(CI; |
| | | | | | | 0.00-0.57, OR = 0) |
| Veillonella | 14 | 22 | 11 | 3 | Yes | P < 0.00001(CI; |
| | | | | | | 0.08-0.30 OR = 0.16) |
| Ruminococcus | 30 | 6 | 14 | 0 | Yes | p-value 0.00007(CI; |
| | | | | | | 0.00-0.21, OR = 0) |
| Sutterella | 7 | 29 | 12 | 2 | Yes | P < 0.00001(CI; |
| | | | | | | 0.01-0.08 OR = 0.03) |
| Prevotella | 10 | 26 | 3 | 11 | No | p-value 0.40(CI; |
| | | | | | | 0.68-2.83, OR = 1.38) |
| Erysiplatoclostridium | 26 | 10 | 12 | 2 | Yes | p-value 0.03(CI; |
| | | | | | | 0.20-0.96, OR = 0.45) |
| Escherichia coli | 21 | 15 | 7 | 7 | No | p-value 0.32(CI; |
| | | | | | | 0.76-2.5, OR = 1.37) |
| Klebsiella | 3 | 33 | 1 | 13 | No | p-value 1 |

According to Tables 13-18, in the 50 patients tested in this study, the relative abundance of Actinobacteria P<0.00001(CI; 0.00-0.3, OR=0) and Proteobacteria bacteria phyla p-value 0.003(CI; 0.00-0.48, OR=0) were significantly less than HC. Although there was not any significant dysbiosis in both Bacteroidetes and Firmicutes phyla. At the class level, there was a significant reduction in Actinobacteria P<0.00001(CI; 0.00-0.07, OR=0), Negativicutes P<0.00001(CI; 0.00-0.21, OR=0), Betaproteobacteria P<0.00001(CI; 0.10-0.46, OR=0.22) and Erysipelotrichia p-value 0.009(CI; 01.27-8.77, OR=3.19).

At the order level, there were significant reductions in more bacteria, including, Bifidobacteriales P<0.00001(CI; 0.00-0.05, OR=0), Veillonellales P<0.00001(CI; 0.08-0.30, OR=0.16), Burkholderiales P<0.00001(CI; 0.11-0.50, OR=0.24), and Erysipelotrichales 0.003(CI; 0.00-0.48, OR=0) and significant increase in Enterobacterales p-value 0.03(CI; 01.04-3.47, OR=3.90) compare to the HC.

At the family levels, Bifidobacteriacae P<0.00001(CI; 0.00-0.05, OR=0), Veilloneacellae P<0.00001(CI; 0.05-0.25, OR=0.12), Sutterellacae p-value 0.0003(CI; 0.16-20.61, OR=0.32), Faecalibacterium p-value 0.00007(CI; 0.00-0.27, OR=0), Prevotellaceae p-value 0.0009(CI; 0.19-0.67, OR=0.36) and Erysipelotrichaceae p-value 0.006(CI; 0.00-0.57, OR=0) showed a significant reduction in Covid-19 patients compare to the HC.

At the genus level, significant reductions in relative abundances of Bifidobacterium P<0.00001(CI; 0.00-0.05, OR=0), Clostridium p-value 0.006(CI; 0.00-0.57, OR=0), Veillonella P<0.00001(CI; 0.08-0.30 OR=0.16). Ruminococcus p-value 0.00007(CI; 0.00-0.21, OR=0), Sutterella P<0.00001(CI; 0.01-0.08 OR=0.03), Faecalibacterium p-value 0.00007(CI; 0.00-0.21, OR=0) and Erysiplatoclostridium p-value 0.03(CI; 0.20-0.96, OR=0.45) and Veillonella P<0.00001(CI; 0.08-0.30 OR=0.16), were observed.

Findings

Significant alterations of the fecal microbiota was observed, in some cases, for the first time, with special baseline characteristics in Covid-19 positive patients. Except for a significantly higher relative abundance of Enterobacterales at order level in Covid-19 positive patients, there was a significant relative abundance reduction in Bifidobacterium, Clostridium. Veillonella, Ruminococcus. Sutterella. Faecalibacterium, Erysiplatoclostridium, and Veillonella at the species and genus levels, Bifidobacteriacae, Veilloneacellae, Sutterellacae, Prevotellaceae and Erysipelotrichaceae at the family level, Bifidobacteriales, Veillonellales, Burkholderiales, and Erysipelotrichales at the order level, Actinobacteria, Negativicutes, Betaproteobacteria, and Erysipelotrichia class level and more importantly, Proteobacteria and Actinobacteria at the phylum level.

There were significant reductions in species, genus, family, order, class, and phylum level between Covid-19 positive patients and the control population. Dysbiosis was largest for Actinobacteria and Proteobacteria, at the phylum level, in the Covid-19 positive populations and must be addressed accordingly. The only significant abundance noted was at Enterobacterales order. At the class level, a significant reduction in Actinobacteria, Negativicutes & Betaproteobacteria was observed. At the order level, significant reductions in more bacteria, including, Bifidobacteriales, Veillonellales, Burkholderiales, and Erysipelotrichales, were observed. At the family levels, Bifidobacteriacae, Veilloneacellae, Sutterellacae, Faecalibacterium, Erysipelotrichales and Prevotellaceae showed a significant reduction in Covid-19 patients compared to the HC. At the genus level, significant reductions in relative abundances of Bifidobacterium, Clostridium, Veillonella, Ruminococcus, Sutterella, Faecalibacterium, and Erysiplatoclostridium were observed.

Interpretation

Covid-19 positive patients will have a considerable reduction in microbiota composition and baseline microbiome dysbiosis, levels of cytokines, and inflammatory markers. Addressing the effects of microbial diversity and consequently, suppression of the immunologic response (known as the cytokine storm) is key to prevent and treat the disease. The importance of reduction at the phylum level must be considered as a possibility of an important indicator for Covid-19 positive susceptible patients. Application of diet-based improvement of microbiome would also be considered as an important preventive measure.

### Example 7: Bifidobacterium and Faecalibacterium Depletion, and Other Bacterial Composition Changes, as Potential Susceptibility Markers of SARS-Cov2 Severity

Objective: To compare gut microbiome diversity and composition in SARS-CoV-2 polymerase chain reaction (PCR)-confirmed positive patients whose symptoms ranged from severe to asymptomatic, and PCR-negative exposed healthy controls.

Summary of Design: Using a cross-sectional study design, shotgun next-generation sequencing (NGS) was sued to evaluate microbiome composition and diversity in patients with PCR-confirmed SARS-CoV-2 infections from March 2020 through January 2021 and SARS-CoV-2 PCR-negative exposed healthy controls. Patients were classified as being asymptomatic or having mild, moderate, or severe symptoms based on NIH criteria. Exposed healthy controls were individuals with prolonged or repeated close contact to patients with SARS-CoV-2 infection or their samples and consisted of family members of subjects or frontline healthcare worker. Microbiome diversity and composition were compared between patients and exposed-controls and across patient subgroups at all taxonomic levels.

Results: 52 patients and 20 controls were identified. Compared with controls, patients had significantly less bacterial diversity (p<0.05) and a lower relative abundance of Bifidobacterium, Faecalibacterium and other bacteria at the genus level. Additionally, there was an inverse association between disease severity and Bifidobacterium (P=0.001) relative abundance and bacterial diversity.

Conclusion: Depletion of Bifidobacteria spp. And Faecalibacterium either before or after infection minimized its immune-dampening effects and allowed SARS-Cov-2 infection to become symptomatic. Thus, low bacterial diversity and low Bifidobacteria abundance is a susceptibility signature for vulnerability to symptoms associated with SARS-CoV-2 infection.

Patients with SARS-CoV-2 infection possess significantly less bacterial diversity and lower abundance of Bifidobacteria and Faecalibacterium and increased abundance of Baceriodies at the genus level compared with exposed-healthy controls. There is an inverse association between disease severity and abundance of Bifidobacteria and Faecalibacterium and direct association of severity and Bacteroides abundance.

Additional Detail Regarding Study Design:

Patients diagnosed with COVID-19 were recruited to the study following expression of interest or physician referral. Informed consent was obtained.

Patients aged ≥18 years of age, with RT-PCR-confirmed SARS-CoV-2 infection within one week of screening were eligible for enrolment. SARS-CoV-2 negative but exposed healthy controls included patients who were PCR-negative for SARS-CoV-2 and remained antibody-negative for between 3-6 months and asymptomatic for 6 months-1 year. SARS-CoV-2 negative exposed healthy controls were further required to meet the following criteria of they either shared a household with at least one symptomatic SARS-CoV-2-positive family member (e.g., sibling, parent, or child) or were a healthcare worker who had been repeatedly directly exposed to symptomatic SARS-CoV-2-positive patients for a minimum of six months or were exposed to numerous SARS-CoV-2-positive patient samples. Also, exposed healthy controls were those that, despite exposure to SARS-CoV-2, chose not to quarantine or take prophylaxis or treatment for COVID-19.

Patients undergoing treatment with total parenteral nutrition, or those with a history of significant gastrointestinal surgery (e.g. bariatric surgery, total colectomy with ileorectal anastomosis, proctocolectomy, postoperative stoma, ostomy, or ileoanal pouch) were excluded.

The study was conducted in accordance with ethical principles of the Declaration of Helsinki, the International Council for Harmonisation (ICH) Harmonised Tripartite Guideline for Good Clinical Practice (GCP), and the E and I Institutional Review Board (IRB) regulations. The clinical study protocol, a sample informed consent form, and other study-related documents were reviewed and approved by the Ethical and Independent Review. All patients provided written informed consent to participate in the study.

Symptomatic patients, as well as asymptomatic household contacts, whose SARS-CoV-2 infectivity was assessed by RT-PCR and exposed healthy controls were required to collect a stool sample. Patients were to collect one mL of fresh stool and place it directly in a Zymo Research DNA/RNA Shield fecal collection tube. Following fecal collection, individual subject DNA was extracted and purified with the Qiagen PowerFecal Pro DNA extraction kit. The isolated DNA was then quantitated utilizing the Quantus Fluorometer with the QuantFluor ONE dsDNA kit. After DNA quantification, the DNA was normalized, and libraries were prepared utilizing shotgun methodology with Illumina's Nextera Flex kit. Per the lab shotgun metagenomic processes, samples underwent tagmentation, amplification, indexing, and purification. After completing the NextSeq run, the raw data were streamed in real-time to Illumina's BaseSpace cloud for conversion to FASTQ files. The FASTQ data were compared to evaluate microbiome diversity in patients with PCR-confirmed SARS-CoV-2 infections and SARS-CoV-2 PCR-negative individuals. The COVID-19 patients had sought remote care in 2020 and 2021. The microbiome differences between the SARS-CoV-2 patients and SARS-CoV-2 exposed healthy controls were compared at all taxonomic levels.

The differences in relative abundance across taxa between the gut microbiome of SARS-CoV-2 infected patients and exposed healthy controls were assessed with One Codex's bioinformatics analysis pipeline using Jupyter notebook in Python. To evaluate the statistical significance of any variability noted between patients and exposed healthy controls at each taxonomic classification, ANOVA and t-test statistics were conducted by R version 3.6.1 (2019-07-05), GraphPad, and SigmaPlot 12.0. Confidence intervals, and p-values were calculated for all comparisons in the statistical analysis, with P-values <0.05 considered significant. Shannon and Simpson indices were calculated by the bioinformatics software One Codex by Python based notebook software.

Patient Characteristics

Demographic and clinical characteristics of patients (n=52) and exposed healthy controls (n=20) are presented in Table 19. Half (50%) of patients and 36% of exposed healthy controls were male. The median (SEM) age of patients was 53±2.54 years and of exposed healthy controls was 48±3.62 years. A total of 85% of patients were non-Hispanic White; 12.5% were Hispanic; and 2.5%, Black and 91% of exposed healthy controls were non-Hispanic White and 9% were Black. Nearly two-thirds of patients (62.5%) had underlying comorbidities considered risk factors for increased morbidity and mortality by CDC.1 Of the SARS-CoV-2 positive individuals, 57.7% had severe disease, 23.1% had moderate disease, and 11.5% had mild disease. The remaining 7.7% were asymptomatic. More than half (54.5%) of exposed controls had underlying comorbidities. The median (SEM) BMI of the 52 patients for whom data were available was 27.9+0.94 compared with 25.1+0.96 for the 20 exposed healthy controls.

Of the healthy exposed subjects, 16 were family members of one of the COVID-19 positive subjects, and 2 were Health Care workers with extensive, non-protected, exposure to subjects and 2 were laboratory personnel exposed to thousands of COVID-19 samples. Given the low number of health care worker subjects, statistics were not performed comparing similarity to family member groups. None of the patients or controls were vaccinated or on COVID-19 prophylaxis or treatment.

TABLE 19

| | | | | | | | PCR | |
|---|---|---|---|---|---|---|---|---|
| No | Age | Sex | Ethnicity | State | Risk Factors | BMI* | Result | Category |
| 1 | 21 | F | W | PA | none | 24.8 | positive | severe |
| 2 | 71 | M | H | CA | none | 18.4 | positive | severe |
| 3 | 86 | M | W | CA | Alzheimer's | 21.1 | positive | severe |
| 4 | 18 | F | W | AZ | none | 22 | positive | severe |
| 5 | 31 | M | W | CO | overweight | 26.9 | positive | severe |
| 6 | 35 | M | W | CA | obese | 30.7 | positive | severe |
| 7 | 43 | F | W | TX | overweight | 25.8 | positive | severe |
| 8 | 70 | F | W | LO | overweight | 25.8 | positive | severe |
| 9 | 56 | M | W | CA | overweight | 27.3 | positive | severe |
| 10 | 44 | F | W | GA | overweight | 27.8 | positive | severe |
| 11 | 53 | M | W | GA | obese | 30.7 | positive | severe |
| 12 | 37 | M | W | FL | hypertension | 23.8 | positive | mild |
| 13 | 60 | M | W | CO | none | 22.7 | positive | severe |
| 14 | 69 | F | W | CO | hypertension | 22.9 | positive | severe |
| 15 | 55 | M | W | NY | obesity, HIV | 37.2 | positive | severe |
| 16 | 67 | F | W | IL | hypertension, | 48.5 | positive | severe |
| | | | | | diabetes | | | |
| | | | | | mellitus, | | | |
| | | | | | obesity, | | | |
| | | | | | cerebral | | | |
| | | | | | aneurysm | | | |
| 17 | 66 | M | W | IL | hypertension, | 33.2 | positive | severe |
| | | | | | obesity, | | | |
| | | | | | pulmonary | | | |
| | | | | | embolus | | | |
| 18 | 49 | F | W | AL | overweight | 27.4 | positive | severe |
| 19 | 60 | M | W | CA | hypertension, | 26.2 | positive | severe |
| | | | | | overweight | | | |
| 20 | 63 | F | W | GA | hypertension, | 28.1 | positive | severe |
| | | | | | overweight | | | |
| 21 | 59 | F | W | GA | hypertension | NA | positive | severe |
| 22 | 53 | F | W | CA | none | 24.5 | positive | severe |
| 23 | 36 | M | W | OR | obese | 30.7 | positive | severe |
| 24 | 10 | M | W | HI | none | 14.5 | positive | severe |
| 25 | 52 | M | W | HI | none | 24.1 | positive | severe |
| 26 | 55 | F | W | HI | hypertension, | 25 | positive | severe |
| | | | | | overweight | | | |
| 27 | 61 | F | W | WA | none | 23.8 | positive | severe |
| 28 | 72 | F | W | CA | none | 19.5 | positive | severe |
| 29 | 62 | M | W | TN | obese, | 31.5 | positive | severe |
| | | | | | hypertension | | | |
| 30 | 18 | M | W | AZ | Crohn's | 15.1 | positive | severe |
| 31 | 23 | F | W | CA | obesity | 30.9 | positive | moderate |
| 32 | 25 | F | W | CA | obesity, | 30.6 | positive | moderate |
| | | | | | asthma | | | |
| 33 | 24 | F | W | AZ | asthma | NA | positive | moderate |
| 34 | 36 | F | H | AZ | hypertension, | 25.4 | positive | moderate |
| | | | | | overweight | | | |
| 35 | 33 | M | H | CA | none | 18.4 | positive | moderate |
| 36 | 63 | F | H | CA | overweight | NA | positive | moderate |
| 37 | 65 | M | W | TX | COPD | NA | positive | moderate |
| 38 | 75 | M | W | CA | hypertension, | 48.5 | positive | moderate |
| | | | | | severe obesity | | | |
| 39 | 67 | M | W | CA | obesity | 33.1 | positive | moderate |
| 40 | 70 | M | W | GA | obesity | 30.7 | positive | moderate |
| 41 | 57 | F | W | TX | obesity | 32 | positive | moderate |
| 42 | 69 | F | W | NE | hypertension | 20.9 | positive | moderate |
| 43 | 19 | M | W | GA | none | 23.5 | positive | mild |
| 44 | 36 | F | W | NY | none | 18.6 | positive | mi |
| 45 | 35 | F | W | CA | overweight | 28.5 | positive | mild |
| 46 | 53 | M | W | NE | overweight | 29 | positive | mild |
| 47 | 38 | F | I | TN | obese | 31.3 | positive | mild |
| 48 | 45 | F | W | PA | hypertension | 23 | positive | mild |
| 49 | 22 | M | W | GA | overweight | 25.8 | positive | asymptomatic |
| 50 | 48 | F | H | CA | none | 20.4 | positive | asymptomatic |
| 51 | 67 | M | W | WI | overweight | 28.5 | positive | asymptomatic |
| 52 | 53 | M | W | IL | obese, | 35.3 | positive | asymptomatic |
| | | | | | diabetes | | | |
| 53 | 68 | F | W | WI | overweight | 26.6 | negative | n/a |
| 54 | 58 | F | H | PA | obesity, | 30 | negative | n/a |
| | | | | | exposed | | | |
| | | | | | to +daughter | | | |
| 55 | 51 | F | W | CA | hypertension, | 25 | negative | n/a |
| | | | | | prediabetes, | | | |
| | | | | | exposed | | | |
| | | | | | to +son, +husband, | | | |
| | | | | | and +daughter | | | |
| 56 | 63 | M | W | CA | overweight | 26.4 | negative | n/a |
| | | | | | exposed | | | |
| | | | | | to +mother | | | |
| 57 | 29 | M | W | CA | overweight: | 26.4 | negative | n/a |
| | | | | | exposed | | | |
| | | | | | to +brother | | | |
| 58 | 23 | F | W | CA | overweight: | 26.4 | negative | n/a |
| | | | | | exposed | | | |
| | | | | | to +brother | | | |
| 59 | 62 | F | W | CA | none: exposed to | 17.4 | negative | n/a |
| | | | | | (kissed) +boyfriend | | | |
| 60 | 51 | F | W | CA | none: exposed | 19.9 | negative | n/a |
| | | | | | to +son, +husband, | | | |
| | | | | | and +daughter | | | |
| 61 | 16 | F | W | CA | none: exposed | 21.2 | negative | n/a |
| | | | | | to +mom | | | |
| 62 | 26 | M | B | CA | none: exposed | 19.5 | negative | n/a |
| | | | | | to +patients | | | |
| 63 | 56 | F | W | CA | overweight: | 25.5 | negative | n/a |
| | | | | | exposed | | | |
| | | | | | to +patient | | | |
| | | | | | samples | | | |
| 64 | 53 | F | W | CA | none: exposed | 24 | negative | n/a |
| | | | | | to workers, | | | |
| | | | | | and +patients | | | |
| | | | | | and +patient | | | |
| | | | | | samples | | | |
| 65 | 35 | M | W | KS | none: exposed | 23.7 | negative | n/a |
| | | | | | to +wife | | | |
| 66 | 35 | M | W | CA | none: exposed | 21.7 | negative | n/a |
| | | | | | to +roommate | | | |
| 67 | 64 | F | H | TN | prediabetic, | 26 | negative | n/a |
| | | | | | overweight | | | |
| 68 | 43 | F | W | CA | overweight, | 26.6 | negative | n/a |
| | | | | | exposed | | | |
| | | | | | to +patient | | | |
| | | | | | samples | | | |
| 69 | 45 | F | W | CA | obese, | 35.1 | negative | n/a |
| | | | | | exposed | | | |
| | | | | | to +patients | | | |
| 70 | 44 | M | W | CA | overweight, | 27.2 | negative | n/a |
| | | | | | exposed | | | |
| | | | | | to +patients | | | |
| | | | | | and +patient | | | |
| | | | | | samples | | | |
| 71 | 52 | M | W | CA | overweight, | 27.1 | negative | n/a |
| | | | | | exposed | | | |
| | | | | | to +patients | | | |
| 72 | 15 | F | W | AZ | none:exposed | 16.9 | negative | n/a |
| | | | | | to +brother | | | |

*BMI, body mass index.

Gut Microbiome Diversity and Composition

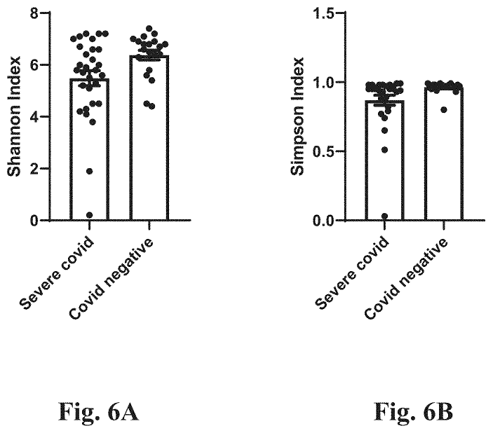

Alpha diversity in patients and exposed healthy controls are shown in FIG. 6A (Difference in Shannon Index for genus and family levels [95% CI 0.11 to 1.67, P=(0.026]) and 1B (Difference in Simpson Index [95% CI 0.0031 to 0.18, P=0.043]) suggesting that the gut microbiome of patients was less diverse than that of exposed healthy controls.

Microbiome analysis revealed significant differences between the two groups. Table 20 lists the bacteria that had significant or otherwise different abundance of bacteria, either comparing difference due to COVID-19 severity (center column) among COVID-19 positive subjects or difference due to COVID-19 positivity (right column) among all subjects. Patients with PCR-confirmed SARS-CoV-2 infection showed a significantly decreased abundance of Bifidobacterium spp., Clostridium, Faecali NR, Faecalibacterium prausnitzii, Ruminococcus, and Subdolingranulum and increased abundance of Bacteriodes. (Table 20 right column). Table 20 left column shows the abundance of bacteria was affected by patient's level of symptom severity. A one-way ANOVA analysis revealed a significant relation between increased disease severity and decrease in abundance of Bifidobacterium spp., Faecali NR. Faecalibacterium prausnitzii, Ruminococcus, and Subdolingranulum and increase in abundance of Bacteriodes and Dorea.

TABLE 20

| | P-value | P-value |
|---|---|---|
| | Does severity of | Does COVID-19 |
| | COVID-19 affect | positivity affect |
| | bacterial | bacterial |
| | abundance? | abundance? |
| Akkermansia | 0.0790 | 0.6933 |
| Alistpies | 0.7860 | 0.5922 |
| Bacteroides | 0.0380 ↑ | 0.0023 ↑ |
| Bifido | 0.0010 ↓ | 0.0001 ↓ |
| Blautia | 0.0800 | 0.2859 |
| Clostridium | 0.8420 | 0.0326 ↓ |
| Collinsella | 0.8080 | 0.9551 |
| Coprococcus | 0.2340 | 0.0607 |
| Dorea | 0.0380 ↑ | 0.3731 |
| Eubacterium | 0.7580 | 0.1825 |
| Faecalibacterium | 0.0100 ↓ | 0.0001 ↓ |
| Faecalibacterium prausnitzii | 0.0070 ↓ | 0.0060 ↓ |
| Klebsiella | 0.8780 | 0.6280 |
| Oscillobacter | 0.8830 | 0.6365 |
| Parabacteroides | 0.4070 | 0.4734 |
| Prevotella | 0.6770 | 0.9510 |
| Roseburia | 0.4030 | 0.1557 |
| Ruminococcus | 0.0900 | 0.0415 ↓ |
| Subdoligranulum | 0.8970 | 0.0058 ↓ |

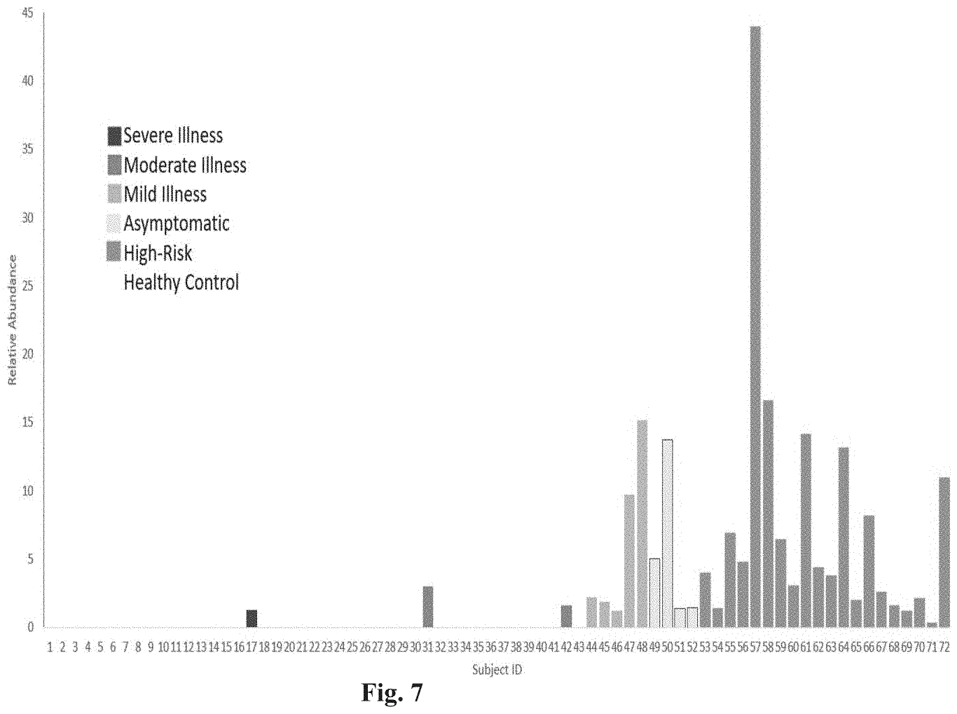

FIG. 7 shows the relative abundance of Bifidobacteria for each subject, grouped by COVID-19 severity. Analyzed via one-way ANOVA with Dunnett's post-hoc (comparing all COVID-19 severities including asymptomatic to healthy controls), there was a significant (P=0.0002) association between severity and Bifidobacteria relative abundance, particularly for severe (P<0.0001) and moderate severity (P=0.0031).

### DISCUSSION

Immune Function and Health could be Enhanced by Bacterial Abundance

Interactions between the host and gut microbiota are complex, numerous, and bidirectional. Gut microbiota regulate the development and function of the innate and adaptive immune system, potentially allowing them to protect against COVID-19. The study's main findings are that COVID-19 severity and positivity correlate to decreased levels of the pro-immune Bifidobacteria and Faecalibacteria and decreased levels of bacterial diversity. This was the first one to compare exposed to SARS-CoV-2 healthy subjects with COVID-19 affected subjects. That is, the following parameters were controlled: COVID-19 exposure, and observing bacterial changes are the associated with development of COVID-19 positivity.

Bacterial diversity is known to inversely relate to presence of various common disorders. This study showed that bacterial diversity indices (Shannon and Simpson Index), show that higher diversity correlates (p<0.05) to less COVID-19 severity, just as it reflects less vulnerability to many other disorders.

The genus Bifidobacterium has important immune functions, is a major component of the microbiome, and is frequently used in probiotics. Bifidobacteria are gram positive non-motile anaerobic rod-shaped bacteria. Bifidobacteria protect against intestinal epithelial cell damage and this protection is independent of their effects on tumor necrosis factor alpha (TNF-α) production. The exopolysaccharide coat which is a feature of some Bifidobacteria has been shown to play a significant role in this protective effect. Bifidobacteria also reduce cell damage by inhibiting TNF-α and macrophages. Also, Bifidobacteria increase Treg responses. These immune functions of Bifidobacteria could be critical for its COVID-protective effect.

Faecalibacterium genus and Faecalibacterium prausnitzii species levels were also inversely related to COVID-19 severity in this analysis. Age and diabetes are risk factors for COVID-19, and F. prausnitzii levels decline markedly in elder and diabetic populations. In fact, Faecalibacterium is considered an “indicator” of human health. The “western” diet (consumption of more meat, animal fat, sugar, processed foods, and low fiber) reduces the level of F. prausnitzii, while a high-fiber (e.g. Mediterranean diet of vegetables and fruits) and low meat diet enhances the count of F. prausnitzii. Preliminary studies show that reduced use of this Mediterranean diet within the same country was associated with increased COVID-19 death. In conclusion, it was shown that F. prausnitzii levels correlate to COVID severity and prior studies show that reduced F prausnitzii is associated with COVID-19 vulnerabilities including age, diabetes, obesity, and possibly diet.

Evidence has accumulated to support a beneficial effect from supplementation with Bifidobacteria in a number of disease states. The number of commensal Bifidobacteria has been shown to decrease with age, a major COVID-19 risk factor. This study found that there is significant relationship between disease severity and relative abundance of Bifidobacterium spp.; patients with a more severe course of viral infection having decreased Bifidobacteria levels. However, it should be noted that there are no definitive studies concerning w % bat constitutes a normal baseline abundance of Bifidobacteria in a “healthy” individual.

Changes in other bacteria were observed, characterized, namely: genus Bifidobacterium, Clostridium, Faecalibacterium (especially species F. prausnitzii), Subdolingranulum, and Ruminococcus showed decreased in bacterial abundance due to COVID-19 positivity, w % bile genus Bacteroides showed increased bacterial abundance. Similarly, Bifidobacterium, Clostridium, and Faecalibacterium showed decreased bacterial abundance with increased COVID-19 severity, while Dorea and Bacteroides showed increased abundance.

Innate Immunity could be Enhanced by Increased Bacterial Level

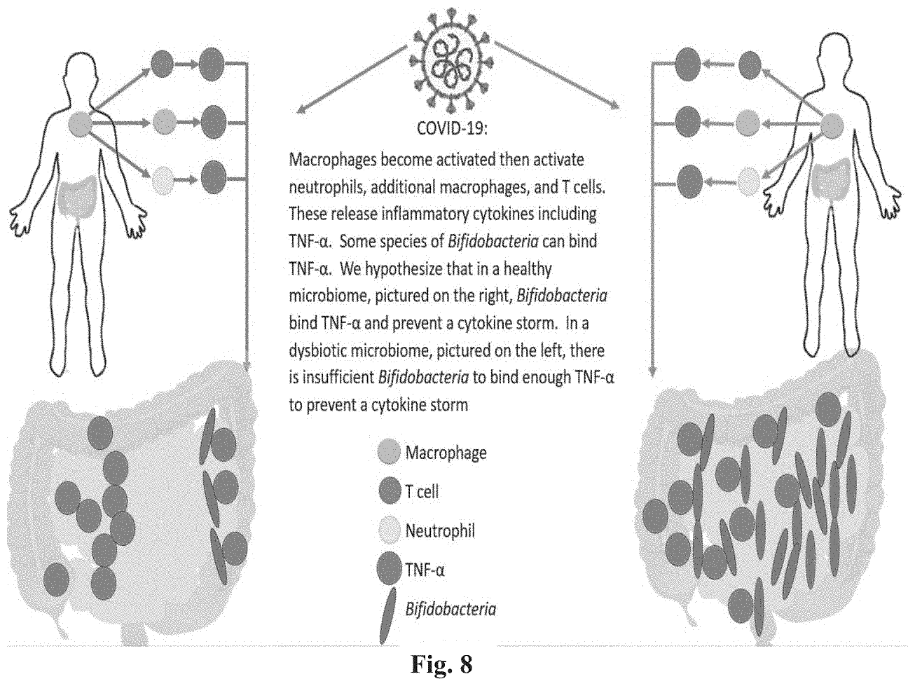

COVID-19 pathology impacts include both direct effects from viral invasion and complex immunological responses including, in its most severe form, the so-called ‘cytokine storm.’ (FIG. 8) The cytokine storm is based upon innate immunity which is also enhanced by Bifidobacteria. Faecalibacteria, and bacterial diversity. Steroid treatment has situational success in COVID-19, and it is based on controlling an overactive innate immunity.

FIG. 8 shows the proposed mechanism for cytokine storm and immune hyper-response in SARS-CoV-2 positive patients. In individuals infected with SARS-CoV-2, the macrophages become activated; these in turn activate T-cells, additional macrophages, and neutrophils—all of which release cytokines, including TNF-α. Bifidobacteria, when present in sufficient numbers, can bind to TNF-α and prevent the subsequent cytokine storm. Therefore, patients with bifidobacterial dysbiosis lack this line of defense which may lead to a cytokine storm.

The findings reported in the current study support the role of commensal Bifidobacteria in the severity of viral infection, suggesting the importance of microbiota in viral pathogenesis and their potential for use in treatment (FIG. 8).

### CONCLUSIONS

Given the cross-sectional study design, it is not possible to determine whether the differences in levels of Bifidobacteria observed between patients and exposed healthy controls preceded or followed infection. If the differences preceded SARS-CoV-2 infection and are a marker of susceptibility, then boosting Bifidobacteria levels is be expected to decrease the risk or severity of SARS-CoV-2 infection. If changes followed SARS-CoV-2 infection, then adjunctive repopulation of the gut microbiome and boosting of Bifidobacteria through supplements or fecal transplant can speed recovery and reduce organ damage; particularly for patients hospitalized with ‘severe’ disease (including children with SARS-CoV-2-related multisystem inflammatory syndrome) and those with persistent symptoms.

COVID-19 ranges in presentation from asymptomatic to fatal, and bacterial composition and diversity also demonstrate marked variation. The same changes observed due to COVID-19, namely drop in Bifidobacterium and/or Faecalibacterium, underly COVID-19 risk factors including obesity, old age, and diabetes. Thus, bacterial levels, especially for Bifidobacterium and/or Faecalibacterium may be markers that determine the response (asymptomatic, severe, fatal) to infection by the same SARS-COV2 virus.

In summary, in this study, findings from only COVID-exposed subjects, both healthy and COVID-19 positive, were presented, and show the level of various bacteria are significantly related to the severity of COVID-19 and SARS-CoV-2 positive patients possessed significantly less bacterial diversity. These findings suggest that Bifidobacterium and other probiotic supplementation or repopulation via fecal transplant may speed recovery and reduce organ damage, particularly for patients hospitalized with severe disease. The data also suggest that characterizing the microbiome may allow the ability to predict vulnerability and appropriately treat COVID-19 patients in a manner dependent on their microbiome.

Accordingly, susceptibility to COVID-19 infection can be determined by determining an amount of Bifidobacterium, Clostridium, Faecalibacterium, Faecalibacterium prausnitzii, Ruminococcus, and Subdoligranulum in the stool sample. The amounts determined to indicate a susceptibility to COVID-19 infection is a decreased abundance of Bifidobacterium, Clostridium, Faecalibacterium, Faecalibacterium prausnitzii, Ruminococcus, and Subdolingranulum and an increased abundance of Bacteroides, as compared to an individual who is less susceptible to COVID-19 infection.

More specifically, COVID-19 susceptibility can be determined by determining an amount of Bifidobacterium and Faecalibacterium in the stool sample. Susceptibility is indicated by a decreased abundance of Bifidobacterium and Faecalibacterium as compared to an individual who is less susceptible to COVID-19 infection.

Shotgun next-generation sequencing is performed on the stool sample to determine the bacteria levels.

The severity of COVID-19 infection in an individual can be reduced by administering at least one of the following to the individual: Bifidobacterium and or Faecalibacterium, and they can be administered to the individual orally, topically, or anally.

The severity of COVID-19 infection in an individual can be reduced by administering Bifidobacterium, Clostridium, Faecalibacterium, Faecalibacterium prausnitzii, and Ruminococcus.

The risk of COVID-19 infection in an individual can be reduced by administering at least one of the following to the individual: Bifidobacterium and or Faecalibacterium, and they can be administered to the individual orally, topically, or anally.

The risk of COVID-19 infection in an individual can be reduced by administering Bifidobacterium, Clostridium, Faecalibacterium, Faecalibacterium prausnitzii, and Ruminococcus.

The amount of Bifidobacterium and Faecalibacterium in an individual can be increased by administering one or more of the following: Vitamin C, Zinc, ivermectin and doxycycline. The vitamin C, zinc, ivermectin and doxycycline can be administered to the individual orally, topically, intravenously, submuscularly or anally.

1,000 mg to 14,000 mg of vitamin C can be administered to the individual at least once to increase the amount of Bifidobacterium and Faecalibacterium in the individual.

25 mg to 75 mg of zinc, 200 mg of doxycycline, and 4 mg to 200 mg of ivermectin can be administered to the individual at least once to increase the amount of Bifidobacterium and Faecalibacterium in the individual.

4 mg to 200 mg of ivermectin is administered to the individual at least once to increase the amount of Bifidobacterium and Faecalibacterium in the individual

Having thus described the invention, it should be apparent that numerous structural modifications and adaptations may be resorted to without departing from the scope and fair meaning of the instant invention as set forth herein above and described herein below by the claims.

What is claimed is:

1. A method of determining susceptibility to COVID-19 infection in an individual, the method comprising the steps of:
a) providing a stool sample from the individual; and
b) determining an amount of Bifidobacterium, Clostridium, Faecalibacterium, Faecalibacterium prausnitzii, Ruminococcus, and Subdoligranulum in the stool sample.

2. The method of claim 1, wherein the amounts determined in step b) comprise a decreased abundance of Bifidobacterium, Clostridium, Faecalibacterium, Faecalibacterium prausnitzii, Ruminococcus, and Subdolingranulum and an increased abundance of Bacteroides, as compared to an individual who is less susceptible to COVID-19 infection.

3. The method of claim 1, wherein the step of determining comprises performing shotgun next-generation sequencing on the stool sample.

4. A method of determining susceptibility to COVID-19 infection in an individual, the method comprising the steps of:
a) providing a stool sample from the individual; and
b) determining an amount of Bifidobacterium and Faecalibacterium in the stool sample.

5. The method of claim 4, wherein the amounts determined in step b) comprise a decreased abundance of Bifidobacterium and Faecalibacterium as compared to an individual who is less susceptible to COVID-19 infection.

6. The method of claim 4, wherein the step of determining comprises performing shotgun next-generation sequencing on the stool sample.

7. A method of reducing the severity of COVID-19 infection in an individual, the method comprising the steps of:
a) providing the individual; and
b) administering at least one of the following to the individual: Bifidobacterium and or Faecalibacterium.

8. The method of claim 7, wherein the step of administering comprises administering the bacterium to the individual orally, topically, or anally.

9. The method of claim 7, wherein the step of administering comprises administering Bifidobacterium, Clostridium, Faecalibacterium, Faecalibacterium prausnitzii, and Ruminococcus,

10. A method of reducing the risk of infection of COVID-19 in an individual, the method comprising the steps of:
a) providing the individual; and
b) administering at least one of the following to the individual: Bifidobacterium and or Faecalibacterium.

11. The method of claim 10, wherein the step of administering comprises administering the bacterium to the individual orally, topically, or anally.

12. The method of claim 10, wherein the step of administering comprises administering Bifidobacterium, Clostridium, Faecalibacterium, Faecalibacterium prausnitzii, and Ruminococcus.

13. A method of increasing an amount of Bifidobacterium and Faecalibacterium in an individual, the method comprising the steps of:
a) providing the individual; and
b) administering one or more of the following: Vitamin C, Zinc, Ivermectin and doxycycline.

14. The method of claim 13, wherein the step of administering comprises administering the vitamin C, zinc, ivermectin and doxycycline to the individual orally, topically, intravenously, submuscularly or anally.

15. The method of claim 13, wherein 1,000 mg to 14,000 mg of vitamin C is administered to the individual at least once.

16. The method of claim 13, wherein 25 mg to 75 mg of zinc, 200 mg of doxycycline, and 4 mg to 200 mg of ivermectin are administered to the individual at least once.

17. The method of claim 13, wherein 4 mg to 200 mg of ivermectin is administered to the individual at least once.
